SEQUENCE LISTING (103 sequences)

SEQ ID NO: 1 - protein, 98 aa, Homo sapiens
MDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVS

SEQ ID NO: 2 - protein, 45 aa, Homo sapiens
RTLVTFKDVFVDFTREEWKLLDTAQQIVYRNVMLENYKNLVSLGY

SEQ ID NO: 3 - protein, 36 aa, Homo sapiens
MAAAVRMNIQMLLEAADYLERREREAEHGYASMLPY

SEQ ID NO: 4 - protein, 58 aa, Homo sapiens
GASQCMPLKLRFKRRWSEDCRLEGGGGPAGGFEDEGEDKKVRGEGPGEAGGPLTPRRV

SEQ ID NO: 5 - protein, 13 aa, Herpes simplex virus 7
DALDDFDLDMLGS

SEQ ID NO: 6 - protein, 55 aa, Artificial Sequence
  Synthetic construct
GRADALDDFDLDMLGSDALDDFDLDMLGSDALDDFDLDMLGSDALDDFDLDMLIN

SEQ ID NO: 7 - protein, 102 aa, Homo sapiens
KGFGAFERSILTQIDHILMDKERLLRRTQTKRSVYRVLGKPEPAAQPVPESLPGEPEILPQAPANAHLKDLDEEIFDDDDFYHQLLRELIERKTSSLDPNDQ

SEQ ID NO: 8 - protein, 31 aa, Homo sapiens
PGLPNGLLSGDEDFSSIADMDFSALLSQISS

SEQ ID NO: 9 - protein, 48 aa, Homo sapiens
PYTPNLPHHQNGHLQHHPPMPPHPGHYWPVHNELAFQPPISNHPAPEY

SEQ ID NO: 10 - protein, 100 aa, Homo sapiens
PPHLNPQDPLKDLVSLACDPASQQPGPLNGSGQLKMPSHCLSAQMLAPPPPGLPRLALPPATKPATTSEGGATSPTSPSYSPPDTSPANRSFVGLGPRDP

SEQ ID NO: 11 - protein, 68 aa, Homo sapiens
ADFQPPYFPPPYQPIYPQSQDPYSHVNDPYSLNPLHAQPQPQHPGWPGQRQSQESGLLHTHRGLPHQL

SEQ ID NO: 12 - protein, 112 aa, Homo sapiens
NRTVSGGQYVVAAAPNLQNQQVLTGLPGVMPNIQYQVIPQFQTVDGQQLQFAATGAQVQQDGSGQIQIIPGANQQIITNRGSGGNIIAAMPNLLQQAVPLQGLANNVLSGQT

SEQ ID NO: 13 - protein, 143 aa, Homo sapiens
QGQTPQRVSGLQGSDALNIQQNQTSGGSLQAGQQKEGEQNQQTQQQQILIQPQLVQGGQALQALQAAPLSGQTFTTQAISQETLQNLQLQAVPNSGPIIIRTPTVGPNGQVSWQTLQLQNLQVQNPQAQTITLAPMQGVSLGQ

SEQ ID NO: 14 - protein, 95 aa, Homo sapiens
DLQQLQQLQQQNLNLQQFVLVHPTTNLQPAQFIISQTPQGQQGLLQAQNLLTQLPQQSQANLLQSQPSITLTSQPATPTRTIAATPIQTLPQSQS

SEQ ID NO: 15 - protein, 63 aa, Homo sapiens
QLAGDIQQLLQLQQLVLVPGHHLQPPAQFLLPQAQQSQPGLLPTPNLFQLPQQTQGALLTSQP

SEQ ID NO: 16 - protein, 90 aa, Artificial Sequence
  synthetic construct
NLFQLPQQTQGALLTSQPNLFQLPQQTQGALLTSQPNLFQLPQQTQGALLTSQPNLFQLPQQTQGALLTSQPNLFQLPQQTQGALLTSQP

SEQ ID NO: 17 - protein, 91 aa, Homo sapiens
PPSTGNSASLSLPLVLQPGLSEPPQPLLPASAPSAPPPAPSLGPGSQQAAFGNPPALLQPPEVPVPHSTQFAANHQEFLPHPQAPQPIVPG

SEQ ID NO: 18 - protein, 111 aa, Homo sapiens
MATRVLSMSARLGPVPQPPAPQDEPVFAQLKPVLGAANPARDAALFPGEELKHAHHRPQAQPAPAQAPQPAQPPATGPRLPPEDLVQTRCEMEKYLTPQLPPVPIIPEHKK

SEQ ID NO: 19 - protein, 88 aa, Homo sapiens
MALSEPILPSFSTFASPCRERGLQERWPRAEPESGGTDDDLNSVLDFILSMGLDGLGAEAAPEPPPPPPPPAFYYPEPGAPPPYSAPA

SEQ ID NO: 20 - protein, 11 aa, Human immunodeficiency virus
YGRKKRRQRRR

SEQ ID NO: 21 - DNA, 1000 bp, Homo sapiens
cgactccccccgggcccaaagtacgtatgcgccgacccccgctatcccgtcccttccctgaagcctccccagagggcgtgtcaggccgcccggccccgagcgcggccgagacgctgcggcaccgtttccgtgcaaccccgtagcccctttcgaagtgacacacttcacgcaactcggcccggcggcggcggcgcgggccactcacgcagctcagccgcgggaggcgccccggctcttgtggcccgcccgctgtcacccgcaggggcactggcggcgcttgccgccaaggggcagagcgagctcccgagtgggtctggagccgcggagctgggcgggggcgggaaggaggtagcgagaaaagaaactggagaaactcggtggccctcttaacgccgccccagagagaccaggtcggcccccgccgctgccgccgccaccctttttcctggggagttgggggcggggggcgaagcgcggcgcaccgggcggggcggccacgccaggggacgcgggcgtgcaggcgccgtcggggccggggtggcggggcccgcgcggagggcgtgggggcagggaccgcgggcgcccctgcagttgccaagcgtcaccaacaggttgcatcgttccccgcggccgccgcgcggcccctcgggcggggagcggccgggggtggagtgggagcgcgtgtgtgcgagtgtgtgcgcgccgtggcgccgcctccacccgctccccgctcggtcccgctcgctcgcccaggccgggctgccctttcgcgtgtccgcgctctcttccctccgccgccgcctcctccattttgcgagctcgtgtctgtgacgggagcccgagtcaccgcctgcccgtcggggacggattctgtgggtggaaggagacgccgcagccggagcggccgaagcagctgggaccgggacggggcacgcgcgcccggaacctcgacccgcggagcccggcgcggggcggagggctggcttgtcagctgggcaat

SEQ ID NO: 22 - DNA, 1000 bp, Homo sapiens
agcaaacgtttacagagctctggacaaaattgagcgcctatgtgtacatggcaagtgtttttagtgtttgtgtgtttacctgcttgtctgggtgattttgcctttgagagtctggatgagaaatgcatggttaaaggcaattccagacaggaagaaaggcagagaagagggtagaaatgacctctgattcttggggctgagggttcctagagcaaatggcacaatgccacgaggcccgatctatccctatgacggaatctaaggtttcagcaagtatctgctggcttggtcatggcttgctcctcagtttgtaggagactctcccactctcccatctgcgcgctcttatcagtcctgaaaagaacccctggcagccaggagcaggtattcctatcgtccttttcctccctccctcgcctccaccctgttggttttttagattgggctttggaaccaaatttggtgagtgctggcctccaggaaatctggagccctggcgcctaaaccttggtttaggaaagcaggagctattcaggaagcaggggtcctccagggctagagctagcctctcctgccctcgcccacgctgcgccagcacttgtttctccaaagccactaggcaggcgttagcgcgcggtgaggggaggggagaaaaggaaaggggaggggagggaaaaggaggtgggaaggcaaggaggccggcccggtgggggcgggacccgactcgcaaactgttgcatttgctctccacctcccagcgccccctccgagatcccggggagccagcttgctgggagagcgggacggtccggagcaagcccagaggcagaggaggcgacagagggaaaaagggccgagctagccgctccagtgctgtacaggagccgaagggacgcaccacgccagccccagcccggctccagcgacagccaacgcctcttgcagcgcggcggcttcgaagccgccgcccggagctgccctttcctcttcggtgaagttt

SEQ ID NO: 23 - DNA, 1000 bp, Homo sapiens
tgcattttaaaaatctgttagctggaccagaccgacaatgtaacataattgccaaagctttggttcgtgacctgaggttatgtttggtatgaaaaggtcacattttatattcagttttctgaagttttggttgcataaccaacctgtggaaggcatgaacacccatgtgcgccctaaccaaaggtttttctgaatcatccttcacatgagaattcctaatgggaccaagtacagtactgtggtccaacataaacacacaagtcaggctgagagaatctcagaaggttgtggaagggtctatctactttgggagcattttgcagaggaagaaactgaggtcctggcaggttgcattctcctgatggcaaaatgcagctcttcctatatgtataccctgaatctccgcccccttcccctcagatgccccctgtcagttcccccagctgctaaatatagctgtctgtggctggctgcgtatgcaaccgcacaccccattctatctgccctatctcggttacagtgtagtcctccccagggtcatcctatgtacacactacgtatttctagccaacgaggagggggaatcaaacagaaagagagacaaacagagatatatcggagtctggcacggggcacataaggcagcacattagagaaagccggcccctggatccgtctttcgcgtttattttaagcccagtcttccctgggccacctttagcagatcctcgtgcgcccccgccccctggccgtgaaactcagcctctatccagcagcgacgacaagtaaagtaaagttcagggaagctgctctttgggatcgctccaaatcgagttgtgcctggagtgatgtttaagccaatgtcagggcaaggcaacagtccctggccgtcctccagcacctttgtaatgcatatgagctcgggagaccagtacttaaagttggaggcccgggagcccaggagctggcggagggcgttcgtcctgggactgcacttgct

SEQ ID NO: 24 - protein, 289 aa, Artificial Sequence
  synthetic construct
MHHHHHHGYGRKKRRQRRRGYPYDVPDYAPCQPMKRLTLGNDIMAAAVRMNIQMLLEAADYLERREREAEHGYASMLPYPKKKRKVGLEPGEKPYKCPECGKSFSDPGHLVRHQRTHTGEKPYKCPECGKSFSRNDALTEHQRTHTGEKPYKCPECGKSFSQSGNLTEHQRTHTGEKPYKCPECGKSFSTSGSLVRHQRTHTGEKPYKCPECGKSFSTSGHLVRHQRTHTGEKPYKCPECGKSFSTSGNLVRHQRTHTGEQKLISEEDLEQKLISEEDLEQKLISEEDL

SEQ ID NO: 25 - protein, 12 aa, Artificial Sequence
  Synthetic construct
PVRRPRRRRRRK

SEQ ID NO: 26 - protein, 12 aa, Artificial Sequence
  Synthetic construct
THRLPRRRRRRK

SEQ ID NO: 27 - protein, 9 aa, Artificial Sequence
  Synthetic construct
RRRRRRRRR

SEQ ID NO: 28 - protein, 16 aa, Drosophila melanogaster
RQILIWFQNRRMKWKK

SEQ ID NO: 29 - DNA, 18 bp, Homo sapiens
cgcgcggagggcgtgggg

SEQ ID NO: 30 - DNA, 18 bp, Homo sapiens
cggggagcggccgggggt

SEQ ID NO: 31 - DNA, 18 bp, Homo sapiens
gcctccacccgctccccg

SEQ ID NO: 32 - DNA, 18 bp, Homo sapiens
ctccagggctagagctag

SEQ ID NO: 33 - DNA, 18 bp, Homo sapiens
ggcccggtgggggcggga

SEQ ID NO: 34 - DNA, 18 bp, Homo sapiens
agcgggacggtccggagc

SEQ ID NO: 35 - DNA, 18 bp, Homo sapiens
gcaggagctattcaggaa

SEQ ID NO: 36 - DNA, 18 bp, Homo sapiens
tccagcagcgacgacaag

SEQ ID NO: 37 - DNA, 18 bp, Homo sapiens
gtccctggccgtcctcca

SEQ ID NO: 38 - DNA, 18 bp, Homo sapiens
gttggaggcccgggagcc

SEQ ID NO: 39 - protein, 168 aa, Artificial Sequence
  synthetic construct
GEKPYKCPECGKSFSRSDKLVRHQRTHTGEKPYKCPECGKSFSRSDELVRHQRTHTGEKPYKCPECGKSFSDPGHLVRHQRTHTGEKPYKCPECGKSFSRSDNLVRHQRTHTGEKPYKCPECGKSFSRSDDLVRHQRTHTGEKPYKCPECGKSFSHTGHLLEHQRTHT

SEQ ID NO: 40 - protein, 168 aa, Artificial Sequence
  synthetic construct
GEKPYKCPECGKSFSTSGHLVRHQRTHTGEKPYKCPECGKSFSRSDKLVRHQRTHTGEKPYKCPECGKSFSDCRDLARHQRTHTGEKPYKCPECGKSFSRSDDLVRHQRTHTGEKPYKCPECGKSFSQRAHLERHQRTHTGEKPYKCPECGKSFSRSDKLTEHQRTHT

SEQ ID NO: 41 - protein, 168 aa, Artificial Sequence
  synthetic construct
GEKPYKCPECGKSFSDPGHLVRHQRTHTGEKPYKCPECGKSFSQRAHLERHQRTHTGEKPYKCPECGKSFSTSGHLVRHQRTHTGEKPYKCPECGKSFSRSDDLVRHQRTHTGEKPYKCPECGKSFSQRAHLERHQRTHTGEKPYKCPECGKSFSRSDKLTEHQRTHT

SEQ ID NO: 42 - protein, 309 aa, Artificial Sequence
  synthetic construct
MHHHHHHGYGRKKRRQRRRGYPYDVPDYAPWDIMPKKKRKVGLEPGEKPYKCPECGKSFSRSDKLVRHQRTHTGEKPYKCPECGKSFSRSDELVRHQRTHTGEKPYKCPECGKSFSDPGHLVRHQRTHTGEKPYKCPECGKSFSRSDNLVRHQRTHTGEKPYKCPECGKSFSRSDDLVRHQRTHTGEKPYKCPECGKSFSHTGHLLEHQRTHTGGGSGGSEFGRADALDDFDLDMLGSDALDDFDLDMLGSDALDDFDLDMLGSDALDDFDLDMLINGSEQKLISEEDLEQKLISEEDLEQKLISEEDL

SEQ ID NO: 43 - protein, 309 aa, Artificial Sequence
  synthetic construct
MHHHHHHGYGRKKRRQRRRGYPYDVPDYAPWDIMPKKKRKVGLEPGEKPYKCPECGKSFSTSGHLVRHQRTHTGEKPYKCPECGKSFSRSDKLVRHQRTHTGEKPYKCPECGKSFSDCRDLARHQRTHTGEKPYKCPECGKSFSRSDDLVRHQRTHTGEKPYKCPECGKSFSQRAHLERHQRTHTGEKPYKCPECGKSFSRSDKLTEHQRTHTGGGSGGSEFGRADALDDFDLDMLGSDALDDFDLDMLGSDALDDFDLDMLGSDALDDFDLDMLINGSEQKLISEEDLEQKLISEEDLEQKLISEEDL

SEQ ID NO: 44 - protein, 309 aa, Artificial Sequence
  synthetic construct
MHHHHHHGYGRKKRRQRRRGYPYDVPDYAPWDIMPKKKRKVGLEPGEKPYKCPECGKSFSDPGHLVRHQRTHTGEKPYKCPECGKSFSQRAHLERHQRTHTGEKPYKCPECGKSFSTSGHLVRHQRTHTGEKPYKCPECGKSFSRSDDLVRHQRTHTGEKPYKCPECGKSFSQRAHLERHQRTHTGEKPYKCPECGKSFSRSDKLTEHQRTHTGGGSGGSEFGRADALDDFDLDMLGSDALDDFDLDMLGSDALDDFDLDMLGSDALDDFDLDMLINGSEQKLISEEDLEQKLISEEDLEQKLISEEDL

SEQ ID NO: 45 - protein, 279 aa, Artificial Sequence
  synthetic construct
MHHHHHHGYGRKKRRQRRRGYPYDVPDYAPWDIMAAAVRMNIQMLLEAADYLERREREAEHGYASMLPYPKKKRKVGLEPGEKPYKCPECGKSFSRSDKLVRHQRTHTGEKPYKCPECGKSFSRSDELVRHQRTHTGEKPYKCPECGKSFSDPGHLVRHQRTHTGEKPYKCPECGKSFSRSDNLVRHQRTHTGEKPYKCPECGKSFSRSDDLVRHQRTHTGEKPYKCPECGKSFSHTGHLLEHQRTHTGEQKLISEEDLEQKLISEEDLEQKLISEEDL

SEQ ID NO: 46 - protein, 279 aa, Artificial Sequence
  synthetic construct
MHHHHHHGYGRKKRRQRRRGYPYDVPDYAPWDIMAAAVRMNIQMLLEAADYLERREREAEHGYASMLPYPKKKRKVGLEPGEKPYKCPECGKSFSTSGHLVRHQRTHTGEKPYKCPECGKSFSRSDKLVRHQRTHTGEKPYKCPECGKSFSDCRDLARHQRTHTGEKPYKCPECGKSFSRSDDLVRHQRTHTGEKPYKCPECGKSFSQRAHLERHQRTHTGEKPYKCPECGKSFSRSDKLTEHQRTHTGEQKLISEEDLEQKLISEEDLEQKLISEEDL

SEQ ID NO: 47 - protein, 279 aa, Artificial Sequence
  synthetic construct
MHHHHHHGYGRKKRRQRRRGYPYDVPDYAPWDIMAAAVRMNIQMLLEAADYLERREREAEHGYASMLPYPKKKRKVGLEPGEKPYKCPECGKSFSDPGHLVRHQRTHTGEKPYKCPECGKSFSQRAHLERHQRTHTGEKPYKCPECGKSFSTSGHLVRHQRTHTGEKPYKCPECGKSFSRSDDLVRHQRTHTGEKPYKCPECGKSFSQRAHLERHQRTHTGEKPYKCPECGKSFSRSDKLTEHQRTHTGEQKLISEEDLEQKLISEEDLEQKLISEEDL

SEQ ID NO: 48 - protein, 168 aa, Artificial Sequence
  synthetic construct
GEKPYKCPECGKSFSRSDNLVRHQRTHTGEKPYKCPECGKSFSRNDALTEHQRTHTGEKPYKCPECGKSFSDCRDLARHQRTHTGEKPYKCPECGKSFSQNSTLTEHQRTHTGEKPYKCPECGKSFSTSGELVRHQRTHTGEKPYKCPECGKSFSQNSTLTEHQRTHT

SEQ ID NO: 49 - protein, 168 aa, Artificial Sequence
  synthetic construct
GEKPYKCPECGKSFSQRAHLERHQRTHTGEKPYKCPECGKSFSRSDDLVRHQRTHTGEKPYKCPECGKSFSRSDKLVRHQRTHTGEKPYKCPECGKSFSRSDELVRHQRTHTGEKPYKCPECGKSFSRNDTLTEHQRTHTGEKPYKCPECGKSFSDPGHLVRHQRTHT

SEQ ID NO: 50 - protein, 168 aa, Artificial Sequence
  synthetic construct
GEKPYKCPECGKSFSTSGELVRHQRTHTGEKPYKCPECGKSFSSKKHLAEHQRTHTGEKPYKCPECGKSFSSRRTCRAHQRTHTGEKPYKCPECGKSFSDPGNLVRHQRTHTGEKPYKCPECGKSFSRNDTLTEHQRTHTGEKPYKCPECGKSFSTSGELVRHQRTHT

SEQ ID NO: 51 - protein, 168 aa, Artificial Sequence
  synthetic construct
GEKPYKCPECGKSFSQRAHLERHQRTHTGEKPYKCPECGKSFSRADNLTEHQRTHTGEKPYKCPECGKSFSHKNALQNHQRTHTGEKPYKCPECGKSFSTSGNLVRHQRTHTGEKPYKCPECGKSFSQRAHLERHQRTHTGEKPYKCPECGKSFSQSSSLVRHQRTHT

SEQ ID NO: 52 - protein, 168 aa, Artificial Sequence
  synthetic construct
GEKPYKCPECGKSFSTSGNLVRHQRTHTGEKPYKCPECGKSFSRADNLTEHQRTHTGEKPYKCPECGKSFSHKNALQNHQRTHTGEKPYKCPECGKSFSTSGSLVRHQRTHTGEKPYKCPECGKSFSQRAHLERHQRTHTGEKPYKCPECGKSFSDPGALVRHQRTHT

SEQ ID NO: 53 - protein, 168 aa, Artificial Sequence
  synthetic construct
GEKPYKCPECGKSFSTSGNLVRHQRTHTGEKPYKCPECGKSFSRADNLTEHQRTHTGEKPYKCPECGKSFSHKNALQNHQRTHTGEKPYKCPECGKSFSQSGDLRRHQRTHTGEKPYKCPECGKSFSQRAHLERHQRTHTGEKPYKCPECGKSFSQSSSLVRHQRTHT

SEQ ID NO: 54 - protein, 309 aa, Artificial Sequence
  Synthetic construct
MHHHHHHGYGRKKRRQRRRGYPYDVPDYAPWDIMPKKKRKVGLEPGEKPYKCPECGKSFSRSDNLVRHQRTHTGEKPYKCPECGKSFSRNDALTEHQRTHTGEKPYKCPECGKSFSDCRDLARHQRTHTGEKPYKCPECGKSFSQNSTLTEHQRTHTGEKPYKCPECGKSFSTSGELVRHQRTHTGEKPYKCPECGKSFSQNSTLTEHQRTHTGGGSGGSEFGRADALDDFDLDMLGSDALDDFDLDMLGSDALDDFDLDMLGSDALDDFDLDMLINGSEQKLISEEDLEQKLISEEDLEQKLISEEDL

SEQ ID NO: 55 - protein, 309 aa, Artificial Sequence
  Synthetic construct
MHHHHHHGYGRKKRRQRRRGYPYDVPDYAPWDIMPKKKRKVGLEPGEKPYKCPECGKSFSQRAHLERHQRTHTGEKPYKCPECGKSFSRSDDLVRHQRTHTGEKPYKCPECGKSFSRSDKLVRHQRTHTGEKPYKCPECGKSFSRSDELVRHQRTHTGEKPYKCPECGKSFSRNDTLTEHQRTHTGEKPYKCPECGKSFSDPGHLVRHQRTHTGGGSGGSEFGRADALDDFDLDMLGSDALDDFDLDMLGSDALDDFDLDMLGSDALDDFDLDMLINGSEQKLISEEDLEQKLISEEDLEQKLISEEDL

SEQ ID NO: 56 - protein, 309 aa, Artificial Sequence
  Synthetic construct
MHHHHHHGYGRKKRRQRRRGYPYDVPDYAPWDIMPKKKRKVGLEPGEKPYKCPECGKSFSTSGELVRHQRTHTGEKPYKCPECGKSFSSKKHLAEHQRTHTGEKPYKCPECGKSFSSRRTCRAHQRTHTGEKPYKCPECGKSFSDPGNLVRHQRTHTGEKPYKCPECGKSFSRNDTLTEHQRTHTGEKPYKCPECGKSFSTSGELVRHQRTHTGGGSGGSEFGRADALDDFDLDMLGSDALDDFDLDMLGSDALDDFDLDMLGSDALDDFDLDMLINGSEQKLISEEDLEQKLISEEDLEQKLISEEDL

SEQ ID NO: 57 - protein, 309 aa, Artificial Sequence
  synthetic construct
MHHHHHHGYGRKKRRQRRRGYPYDVPDYAPWDIMPKKKRKVGLEPGEKPYKCPECGKSFSQRAHLERHQRTHTGEKPYKCPECGKSFSRADNLTEHQRTHTGEKPYKCPECGKSFSHKNALQNHQRTHTGEKPYKCPECGKSFSTSGNLVRHQRTHTGEKPYKCPECGKSFSQRAHLERHQRTHTGEKPYKCPECGKSFSQSSSLVRHQRTHTGGGSGGSEFGRADALDDFDLDMLGSDALDDFDLDMLGSDALDDFDLDMLGSDALDDFDLDMLINGSEQKLISEEDLEQKLISEEDLEQKLISEEDL

SEQ ID NO: 58 - protein, 309 aa, Artificial Sequence
  synthetic construct
MHHHHHHGYGRKKRRQRRRGYPYDVPDYAPWDIMPKKKRKVGLEPGEKPYKCPECGKSFSTSGNLVRHQRTHTGEKPYKCPECGKSFSRADNLTEHQRTHTGEKPYKCPECGKSFSHKNALQNHQRTHTGEKPYKCPECGKSFSTSGSLVRHQRTHTGEKPYKCPECGKSFSQRAHLERHQRTHTGEKPYKCPECGKSFSDPGALVRHQRTHTGGGSGGSEFGRADALDDFDLDMLGSDALDDFDLDMLGSDALDDFDLDMLGSDALDDFDLDMLINGSEQKLISEEDLEQKLISEEDLEQKLISEEDL

SEQ ID NO: 59 - protein, 309 aa, Artificial Sequence
  synthetic construct
MHHHHHHGYGRKKRRQRRRGYPYDVPDYAPWDIMPKKKRKVGLEPGEKPYKCPECGKSFSTSGNLVRHQRTHTGEKPYKCPECGKSFSRADNLTEHQRTHTGEKPYKCPECGKSFSHKNALQNHQRTHTGEKPYKCPECGKSFSQSGDLRRHQRTHTGEKPYKCPECGKSFSQRAHLERHQRTHTGEKPYKCPECGKSFSQSSSLVRHQRTHTGGGSGGSEFGRADALDDFDLDMLGSDALDDFDLDMLGSDALDDFDLDMLGSDALDDFDLDMLINGSEQKLISEEDLEQKLISEEDLEQKLISEEDL

SEQ ID NO: 60 - protein, 279 aa, Artificial Sequence
  synthetic construct
MHHHHHHGYGRKKRRQRRRGYPYDVPDYAPWDIMAAAVRMNIQMLLEAADYLERREREAEHGYASMLPYPKKKRKVGLEPGEKPYKCPECGKSFSRSDNLVRHQRTHTGEKPYKCPECGKSFSRNDALTEHQRTHTGEKPYKCPECGKSFSDCRDLARHQRTHTGEKPYKCPECGKSFSQNSTLTEHQRTHTGEKPYKCPECGKSFSTSGELVRHQRTHTGEKPYKCPECGKSFSQNSTLTEHQRTHTGEQKLISEEDLEQKLISEEDLEQKLISEEDL

SEQ ID NO: 61 - protein, 279 aa, Artificial Sequence
  synthetic construct
MHHHHHHGYGRKKRRQRRRGYPYDVPDYAPWDIMAAAVRMNIQMLLEAADYLERREREAEHGYASMLPYPKKKRKVGLEPGEKPYKCPECGKSFSQRAHLERHQRTHTGEKPYKCPECGKSFSRSDDLVRHQRTHTGEKPYKCPECGKSFSRSDKLVRHQRTHTGEKPYKCPECGKSFSRSDELVRHQRTHTGEKPYKCPECGKSFSRNDTLTEHQRTHTGEKPYKCPECGKSFSDPGHLVRHQRTHTGEQKLISEEDLEQKLISEEDLEQKLISEEDL

SEQ ID NO: 62 - protein, 279 aa, Artificial Sequence
  synthetic construct
MHHHHHHGYGRKKRRQRRRGYPYDVPDYAPWDIMAAAVRMNIQMLLEAADYLERREREAEHGYASMLPYPKKKRKVGLEPGEKPYKCPECGKSFSTSGELVRHQRTHTGEKPYKCPECGKSFSSKKHLAEHQRTHTGEKPYKCPECGKSFSSRRTCRAHQRTHTGEKPYKCPECGKSFSDPGNLVRHQRTHTGEKPYKCPECGKSFSRNDTLTEHQRTHTGEKPYKCPECGKSFSTSGELVRHQRTHTGEQKLISEEDLEQKLISEEDLEQKLISEEDL

SEQ ID NO: 63 - protein, 279 aa, Artificial Sequence
  synthetic construct
MHHHHHHGYGRKKRRQRRRGYPYDVPDYAPWDIMAAAVRMNIQMLLEAADYLERREREAEHGYASMLPYPKKKRKVGLEPGEKPYKCPECGKSFSQRAHLERHQRTHTGEKPYKCPECGKSFSRADNLTEHQRTHTGEKPYKCPECGKSFSHKNALQNHQRTHTGEKPYKCPECGKSFSTSGNLVRHQRTHTGEKPYKCPECGKSFSQRAHLERHQRTHTGEKPYKCPECGKSFSQSSSLVRHQRTHTGEQKLISEEDLEQKLISEEDLEQKLISEEDL

SEQ ID NO: 64 - protein, 279 aa, Artificial Sequence
  synthetic construct
MHHHHHHGYGRKKRRQRRRGYPYDVPDYAPWDIMAAAVRMNIQMLLEAADYLERREREAEHGYASMLPYPKKKRKVGLEPGEKPYKCPECGKSFSTSGNLVRHQRTHTGEKPYKCPECGKSFSRADNLTEHQRTHTGEKPYKCPECGKSFSHKNALQNHQRTHTGEKPYKCPECGKSFSTSGSLVRHQRTHTGEKPYKCPECGKSFSQRAHLERHQRTHTGEKPYKCPECGKSFSDPGALVRHQRTHTGEQKLISEEDLEQKLISEEDLEQKLISEEDL

SEQ ID NO: 65 - protein, 279 aa, Artificial Sequence
  synthetic construct
MHHHHHHGYGRKKRRQRRRGYPYDVPDYAPWDIMAAAVRMNIQMLLEAADYLERREREAEHGYASMLPYPKKKRKVGLEPGEKPYKCPECGKSFSTSGNLVRHQRTHTGEKPYKCPECGKSFSRADNLTEHQRTHTGEKPYKCPECGKSFSHKNALQNHQRTHTGEKPYKCPECGKSFSQSGDLRRHQRTHTGEKPYKCPECGKSFSQRAHLERHQRTHTGEKPYKCPECGKSFSQSSSLVRHQRTHTGEQKLISEEDLEQKLISEEDLEQKLISEEDL

SEQ ID NO: 66 - protein, 168 aa, Artificial Sequence
  synthetic construct
GEKPYKCPECGKSFSQRAHLERHQRTHTGEKPYKCPECGKSFSTSGELVRHQRTHTGEKPYKCPECGKSFSTSGELVRHQRTHTGEKPYKCPECGKSFSDPGALVRHQRTHTGEKPYKCPECGKSFSDPGALVRHQRTHTGEKPYKCPECGKSFSTTGALTEHQRTHT

SEQ ID NO: 67 - protein, 168 aa, Artificial Sequence
  synthetic construct
GEKPYKCPECGKSFSTSHSLTEHQRTHTGEKPYKCPECGKSFSTKNSLTEHQRTHTGEKPYKCPECGKSFSSRRTCRAHQRTHTGEKPYKCPECGKSFSDPGHLVRHQRTHTGEKPYKCPECGKSFSTKNSLTEHQRTHTGEKPYKCPECGKSFSDPGALVRHQRTHT

SEQ ID NO: 68 - protein, 168 aa, Artificial Sequence
  synthetic construct
GEKPYKCPECGKSFSDCRDLARHQRTHTGEKPYKCPECGKSFSQRAHLERHQRTHTGEKPYKCPECGKSFSRNDTLTEHQRTHTGEKPYKCPECGKSFSDPGHLVRHQRTHTGEKPYKCPECGKSFSQRAHLERHQRTHTGEKPYKCPECGKSFSTSGSLVRHQRTHT

SEQ ID NO: 69 - protein, 309 aa, Artificial Sequence
  Synthetic construct
MHHHHHHGYGRKKRRQRRRGYPYDVPDYAPWDIMPKKKRKVGLEPGEKPYKCPECGKSFSQRAHLERHQRTHTGEKPYKCPECGKSFSTSGELVRHQRTHTGEKPYKCPECGKSFSTSGELVRHQRTHTGEKPYKCPECGKSFSDPGALVRHQRTHTGEKPYKCPECGKSFSDPGALVRHQRTHTGEKPYKCPECGKSFSTTGALTEHQRTHTGGGSGGSEFGRADALDDFDLDMLGSDALDDFDLDMLGSDALDDFDLDMLGSDALDDFDLDMLINGSEQKLISEEDLEQKLISEEDLEQKLISEEDL

SEQ ID NO: 70 - protein, 309 aa, Artificial Sequence
  Synthetic construct
MHHHHHHGYGRKKRRQRRRGYPYDVPDYAPWDIMPKKKRKVGLEPGEKPYKCPECGKSFSTSHSLTEHQRTHTGEKPYKCPECGKSFSTKNSLTEHQRTHTGEKPYKCPECGKSFSSRRTCRAHQRTHTGEKPYKCPECGKSFSDPGHLVRHQRTHTGEKPYKCPECGKSFSTKNSLTEHQRTHTGEKPYKCPECGKSFSDPGALVRHQRTHTGGGSGGSEFGRADALDDFDLDMLGSDALDDFDLDMLGSDALDDFDLDMLGSDALDDFDLDMLINGSEQKLISEEDLEQKLISEEDLEQKLISEEDL

SEQ ID NO: 71 - protein, 309 aa, Artificial Sequence
  Synthetic construct
MHHHHHHGYGRKKRRQRRRGYPYDVPDYAPWDIMPKKKRKVGLEPGEKPYKCPECGKSFSDCRDLARHQRTHTGEKPYKCPECGKSFSQRAHLERHQRTHTGEKPYKCPECGKSFSRNDTLTEHQRTHTGEKPYKCPECGKSFSDPGHLVRHQRTHTGEKPYKCPECGKSFSQRAHLERHQRTHTGEKPYKCPECGKSFSTSGSLVRHQRTHTGGGSGGSEFGRADALDDFDLDMLGSDALDDFDLDMLGSDALDDFDLDMLGSDALDDFDLDMLINGSEQKLISEEDLEQKLISEEDLEQKLISEEDL

SEQ ID NO: 72 - protein, 279 aa, Artificial Sequence
  synthetic construct
MHHHHHHGYGRKKRRQRRRGYPYDVPDYAPWDIMAAAVRMNIQMLLEAADYLERREREAEHGYASMLPYPKKKRKVGLEPGEKPYKCPECGKSFSQRAHLERHQRTHTGEKPYKCPECGKSFSTSGELVRHQRTHTGEKPYKCPECGKSFSTSGELVRHQRTHTGEKPYKCPECGKSFSDPGALVRHQRTHTGEKPYKCPECGKSFSDPGALVRHQRTHTGEKPYKCPECGKSFSTTGALTEHQRTHTGEQKLISEEDLEQKLISEEDLEQKLISEEDL

SEQ ID NO: 73 - protein, 279 aa, Artificial Sequence
  synthetic construct
MHHHHHHGYGRKKRRQRRRGYPYDVPDYAPWDIMAAAVRMNIQMLLEAADYLERREREAEHGYASMLPYPKKKRKVGLEPGEKPYKCPECGKSFSTSHSLTEHQRTHTGEKPYKCPECGKSFSTKNSLTEHQRTHTGEKPYKCPECGKSFSSRRTCRAHQRTHTGEKPYKCPECGKSFSDPGHLVRHQRTHTGEKPYKCPECGKSFSTKNSLTEHQRTHTGEKPYKCPECGKSFSDPGALVRHQRTHTGEQKLISEEDLEQKLISEEDLEQKLISEEDL

SEQ ID NO: 74 - protein, 279 aa, Artificial Sequence
  synthetic construct
MHHHHHHGYGRKKRRQRRRGYPYDVPDYAPWDIMAAAVRMNIQMLLEAADYLERREREAEHGYASMLPYPKKKRKVGLEPGEKPYKCPECGKSFSDCRDLARHQRTHTGEKPYKCPECGKSFSQRAHLERHQRTHTGEKPYKCPECGKSFSRNDTLTEHQRTHTGEKPYKCPECGKSFSDPGHLVRHQRTHTGEKPYKCPECGKSFSQRAHLERHQRTHTGEKPYKCPECGKSFSTSGSLVRHQRTHTGEQKLISEEDLEQKLISEEDLEQKLISEEDL

SEQ ID NO: 75 - protein, 7 aa, Simian virus 40
PKKKRKV

SEQ ID NO: 76 - protein, 6 aa, Artificial Sequence
  Synthetic construct
GGSGGS

SEQ ID NO: 77 - DNA, 4513 bp, Artificial Sequence
  Synthetic construct
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtaccgtatacctcgagcccggggaaaagccatataaatgccccgagtgcggcaaatcattcagccaaagtagcaacttagtaagacaccagcgcacccataccggtaagaaaactagtcttaagctcgagcccggggaaaaaccctataaatgccccgagtgtggtaagtcattctctcaaagcggggatttaagaagacaccagagaacccacaccggtaagaaaactagtggcgcgccctcgagcccggggagaaaccttataaatgcccagaatgcgggaaatcgttcagtcaaagagcacatttagaaagacatcaacggacccacaccggtaagaaaactagtcctaggctcgagcccggggaaaaaccttacaagtgccctgagtgcggcaagagcttctctcaatcaagttcattagtaagacaccagaggactcataccggtaagaaaactagtcctcagcctcgagcccggggagaagccttataagtgccctgagtgtggcaaaagcttcagcgatcctggaaatttagtaagacaccaacgcacccacaccggtaagaaaactagtatgcatctcgagcccggggaaaaaccgtataaatgtcctgagtgcggtaagtctttttccgactgtagagacttagcgagacaccaacgtactcataccggtaaaaagactagttgtacactcgagcccggggaaaaaccgtacaagtgtcctgagtgcgggaagagtttctccgatccgggccacttagtaagacatcagaggacacataccggtaaaaagactagtttcgaactcgagcccggggagaaaccatacaaatgccccgagtgtggaaagtcatttagtgatccaggcgcattagtaagacatcagcggacacataccggtaagaaaactagtgaattcctcgagcccggggagaagccatataaatgtcccgagtgtggcaagtccttttctagatcagataatttagtaagacatcagagaacgcacaccggtaaaaagactagtcaattgctcgagcccggggagaagccatacaagtgtcccgaatgcgggaagtcattctccagaagtgacgatttagtaagacatcagcgcacgcacaccggtaagaaaactagtccatggctcgagcccggggagaagccctacaagtgtccagaatgcggaaagagtttctccagaagtgacaaattagtaagacaccagagaacccataccggtaagaaaactagtcatatgctcgagcccggggagaagccgtacaagtgccctgaatgtggtaagtcattttcgagaagtgatgaattagtaagacaccagcggactcataccggtaaaaagactagtgctagcctcgagcccggggagaagccctataaatgtccagaatgtggaaagtcctttagcacgtcagggaacttagtaagacaccagcgaactcataccggtaagaaaactagtttaattaactcgagcccggggagaaaccatacaagtgtccagagtgcgggaaaagctttagtacaagcggtgagttagtaagacaccaacgaacacacaccggtaaaaagactagtgtttaaacctcgagcccggggaaaagccctacaagtgcccggaatgcggcaagtcttttagcaccagcggacatttagtaagacaccagagaacccacaccggtaaaaagactagtccgcggctcgagcccggggaaaagccctacaagtgtcctgagtgcggaaagtctttctccactagcggttcattagtaagacaccagaggacacacaccggtaaaaagactagtgcatgcgtcgactgcagaggcctgcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 78 - DNA, 4442 bp, Artificial Sequence
  Synthetic construct
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacctcgcgaatgcatctagatgtatacctcgagcccggggagaagccctataaatgccctgaatgcgggaaatctttctcttctaagaaggcactcacagaacaccagcggacacacaccggtaaaaaaactagtcttaagctcgagcccggggaaaagccctacaagtgccccgaatgcgggaagtcttttagtcagagtggaaatcttaccgagcaccagagaacacacaccggtaagaagactagtggcgcgccctcgagcccggggagaagccatacaagtgccctgaatgtggcaagtccttttcaagagccgataacctgacagaacaccaaaggacgcataccggtaagaaaactagtcctaggctcgagcccggggagaagccctataaatgccctgaatgtggcaagagcttcagtactagcgggaatctcactgaacatcagcgaactcataccggtaaaaaaactagtcctcagcctcgagcccggggaaaaaccatacaagtgccctgagtgcggcaagagttttagtacctcacactctcttacagaacatcagcgaacccacaccggtaaaaaaactagtatgcatctcgagcccggggagaaaccatacaaatgtcccgaatgtggcaagagtttcagcagtaaaaagcatctcgctgagcatcagagaactcacaccggtaaaaagactagttgtacactcgagcccggggaaaagccctacaaatgccccgaatgtggtaagtctttttctaggaacgacaccttgacagaacaccagcggacccacaccggtaagaagactagtgaattcctcgagcccggggagaagccttataagtgccccgaatgtggaaagagtttctctactaagaatagcctgaccgagcaccagcgcactcacaccggtaagaaaactagtcaattgctcgagcccggggagaagccctataaatgccctgaatgcgggaaatctttctctcaatcaggccacctcacagaacaccagcggacacacaccggtaaaaaaactagtccatggctcgagcccggggagaaaccctataagtgtcccgaatgcgggaaatcattctctcatacagggcatctgctcgaacatcaaaggacgcacaccggtaaaaagactagtcatatgctcgagcccggggaaaagccttacaaatgccccgaatgtgggaagagtttcagccggtctgataagctgaccgaacaccagagaactcataccggtaaaaaaactagtgctagcctcgagcccggggaaaagccctacaagtgccctgagtgtgggaagtccttttcttcaagacgcacgtgccgcgctcaccagcggacacataccggtaagaaaactagtttaattaactcgagcccggggagaaaccatacaaatgtcccgaatgtggcaagtccttctcacagaactctactttgaccgagcatcagagaactcacaccggtaagaagactagtccgcggctcgagcccggggaaaagccttataagtgccccgaatgcggaaagagcttctcaaggaatgatgcacttaccgagcatcaaaggactcataccggtaaaaaaactagtgcatgcttcgaactcgagcccggggaaaagccctataagtgtcccgaatgcggcaagagttttagtactactggcgcactcacagaacaccagcgcactcacaccggtaagaaaactagtgaaagtcctctccactgactgtagcctccaattcactggagatctgacacaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 79 - DNA, 4376 bp, Artificial Sequence
  synthetic construct
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtaccgtatacctcgagcccggggagaagccatacaaatgccctgagtgtggaaagtcatttagccagcgagctaatctgcgggcccaccagcggacccacaccggtaagaagactagtcttaagctcgagcccggggagaagccatacaaatgtccagaatgtggaaagtccttctctgatagtggcaacctcagagtgcatcagcgaacacataccggtaagaagactagtggcgcgccctcgagcccggggaaaagccatataagtgccctgagtgtggaaagagcttcagtaggaaggataaccttaaaaaccaccaaagaacccacaccggtaagaagactagtcctaggctcgagcccggggaaaagccatataaatgtcccgagtgcggcaaatccttctctaccactggcaacctcacagtgcatcaacggactcacaccggtaaaaagactagtcctcagcctcgagcccggggaaaagccctataaatgtcccgagtgcggaaagtctttttccagccctgccgacctgacacgccaccaacgaacgcacaccggtaagaagactagtatgcatctcgagcccggggaaaagccgtacaaatgtccagagtgtggaaaatccttttctgataaaaaggacctgacacggcatcagcgaacccacaccggtaaaaagactagttgtacactcgagcccggggagaaaccttataaatgcccagaatgcggtaaaagtttcagcaggacggataccttgcgggatcatcagagaacccacaccggtaaaaaaactagtgaattcctcgagcccggggaaaaaccatacaagtgccccgagtgtggcaagagctttagtacccacctcgacctgattagacaccagcgcacccacaccggtaagaaaactagtcaattgctcgagcccggggaaaagccctataagtgcccagagtgcgggaaatcattctcacagctggcacatcttagagcccaccagcggacccacaccggtaagaagactagtccatggctcgagcccggggagaaaccctataagtgccctgaatgcggcaagtctttcagtgagcggtcacatctccgagagcaccagcgaacgcacaccggtaaaaagactagtcatatgctcgagcccggggaaaaaccctacaagtgccctgagtgtggaaagtcatttagtcgctccgaccacctgaccaaccatcagcggactcacaccggtaagaaaactagtgctagcctcgagcccggggagaaaccttacaagtgccccgagtgcggcaagagtttcagccacaggaccaccctgacaaaccaccagaggacccacaccggtaaaaagactagtttaattaactcgagcccggggagaaaccttataagtgtcctgagtgcggcaaaagtttctctcaaaagtcctcccttattgcccatcaaaggacccataccggtaagaagactagtgtttaaacctcgagcccggggagaagccctataaatgtcccgagtgcggaaagtccttctcacggcgcgatgaattgaacgtccatcagagaacacacaccggtaaaaaaactagtccgcggctcgagcccggggaaaaaccttataagtgtcccgagtgcggcaagagtttcagtcacaaaaacgcacttcagaatcatcagaggacacataccggtaagaaaactagtgcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 80 - DNA, 57 bp, Artificial Sequence
  Synthetic construct
tcgacaggcccaggcggccctcgaggatatcatgatgactagtggccaggccggccc

SEQ ID NO: 81 - DNA, 57 bp, Artificial Sequence
  Synthetic construct
aattgggccggcctggccactagtcatcatgatatcctcgagggccgcctgggcctg

SEQ ID NO: 82 - DNA, 6699 bp, Artificial Sequence
  Synthetic construct
gcttgcatgcaacttcttttctttttttttcttttctctctcccccgttgttgtctcaccatatccgcaatgacaaaaaaaatgatggaagacactaaaggaaaaaattaacgacaaagacagcaccaacagatgtcgttgttccagagctgatgaggggtatcttcgaacacacgaaactttttccttccttcattcacgcacactactctctaatgagcaacggtatacggccttccttccagttacttgaatttgaaataaaaaaagtttgccgctttgctatcaagtataaatagacctgcaattattaatcttttgtttcctcgtcattgttctcgttccctttcttccttgtttctttttctgcacaatatttcaagctataccaagcatacaatcaactccaagctttgcaaagatggataaagcggaattaattcccgagcctccaaaaaagaagagaaaggtcgaattgggtaccgccgccaattttaatcaaagtgggaatattgctgatagctcattgtccttcactttcactaacagtagcaacggtccgaacctcataacaactcaaacaaattctcaagcgctttcacaaccaattgcctcctctaacgttcatgataacttcatgaataatgaaatcacggctagtaaaattgatgatggtaataattcaaaaccactgtcacctggttggacggaccaaactgcgtataacgcgtttggaatcactacagggatgtttaataccactacaatggatgatgtatataactatctattcgatgatgaagataccccaccaaacccaaaaaaagagatctctcgacaggcccaggcggccctcgaggatatcatgatgactagtggccaggccggcccaattccagatctatgaatcgtagatactgaaaaaccccgcaagttcacttcaactgtgcatcgtgcaccatctcaatttctttcatttatacatcgttttgccttcttttatgtaactatactcctctaagtttcaatcttggccatgtaacctctgatctatagaattttttaaatgactagaattaatgcccatcttttttttggacctaaattcttcatgaaaatatattacgagggcttattcagaagctttggacttcttcgccagaggtttggtcaagtctccaatcaaggttgtcggcttgtctaccttgccagaaatttacgaaaagatggaaaagggtcaaatcgttggtagatacgttgttgacacttctaaataagcgaatttcttatgatttatgatttttattattaaataagttataaaaaaaataagtgtatacaaattttaaagtgactcttaggttttaaaacgaaaattcttattcttgagtaactctttcctgtaggtcaggttgctttctcaggtatagcatgaggtcgctcttattgaccacacctctaccggcatgccggtcgaaattcccctaccctatgaacatattccattttgtaatttcgtgtcgtttctattatgaatttcatttataaagtttatgtacaaatatcataaaaaaagagaatctttttaagcaaggattttcttaacttcttcggcgacagcatcaccgacttcggtggtactgttggaaccacctaaatcaccagttctgatacctgcatccaaaacctttttaactgcatcttcaatggccttaccttcttcaggcaagttcaatgacaatttcaacatcattgcagcagacaagatagtggcgatagggtcaaccttattctttggcaaatctggagcagaaccgtggcatggttcgtacaaaccaaatgcggtgttcttgtctggcaaagaggccaaggacgcagatggcaacaaacccaaggaacctgggataacggaggcttcatcggagatgatatcaccaaacatgttgctggtgattataataccatttaggtgggttgggttcttaactaggatcatggcggcagaatcaatcaattgatgttgaaccttcaatgtaggaaattcgttcttgatggtttcctccacagtttttctccataatcttgaagaggccaaaacattagctttatccaaggaccaaataggcaatggtggctcatgttgtagggccatgaaagcggccattcttgtgattctttgcacttctggaacggtgtattgttcactatcccaagcgacaccatcaccatcgtcttcctttctcttaccaaagtaaatacctcccactaattctctgacaacaacgaagtcagtacctttagcaaattgtggcttgattggagataagtctaaaagagagtcggatgcaaagttacatggtcttaagttggcgtacaattgaagttctttacggatttttagtaaaccttgttcaggtctaacactacctgtaccccatttaggaccacccacagcacctaacaaaacggcatcaaccttcttggaggcttccagcgcctcatctggaagtgggacacctgtagcatcgatagcagcaccaccaattaaatgattttcgaaatcgaacttgacattggaacgaacatcagaaatagctttaagaaccttaatggcttcggctgtgatttcttgaccaacgtggtcacctggcaaaacgacgatcttcttaggggcagacattagaatggtatatccttgaaatatatatatatattgctgaaatgtaaaaggtaagaaaagttagaaagtaagacgattgctaaccacctattggaaaaaacaataggtccttaaataatattgtcaacttcaagtattgtgatgcaagcatttagtcatgaacgcttctctattctatatgaaaagccggttccggcctctcacctttcctttttctcccaatttttcagttgaaaaaggtatatgcgtcaggcgacctctgaaattaacaaaaaatttccagtcatcgaatttgattctgtgcgatagcgcccctgtgtgttctcgttatgttgaggaaaaaaataatggttgctaagagattcgaactcttgcatcttacgatacctgagtattcccacagttggggatctcgactctagctagaggatcaattcgtaatcatggtcatagctgtttcctgtgtgaaattgttatccgctcacaattccacacaacatacgagccggaagcataaagtgtaaagcctggggtgcctaatgagtgaggtaactcacattaattgcgttgcgctcactgcccgctttccagtcgggaaacctgtcgtgccagctggattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataacgcatttaagcataaacacgcactatgccgttcttctcatgtatatatatatacaggcaacacgcagatataggtgcgacgtgaacagtgagctgtatgtgcgcagctcgcgttgcattttcggaagcgctcgttttcggaaacgctttgaagttcctattccgaagttcctattctctagctagaaagtataggaacttcagagcgcttttgaaaaccaaaagcgctctgaagacgcactttcaaaaaaccaaaaacgcaccggactgtaacgagctactaaaatattgcgaataccgcttccacaaacattgctcaaaagtatctctttgctatatatctctgtgctatatccctatataacctacccatccacctttcgctccttgaacttgcatctaaactcgacctctacattttttatgtttatctctagtattactctttagacaaaaaaattgtagtaagaactattcatagagtgaatcgaaaacaatacgaaaatgtaaacatttcctatacgtagtatatagagacaaaatagaagaaaccgttcataattttctgaccaatgaagaatcatcaacgctatcactttctgttcacaaagtatgcgcaatccacatcggtatagaatataatcggggatgcctttatcttgaaaaaatgcacccgcagcttcgctagtaatcagtaaacgcgggaagtggagtcaggctttttttatggaagagaaaatagacaccaaagtagccttcttctaaccttaacggacctacagtgcaaaaagttatcaagagactgcattatagagcgcacaaaggagaaaaaaagtaatctaagatgctttgttagaaaaatagcgctctcgggatgcatttttgtagaacaaaaaagaagtatagattctttgttggtaaaatagcgctctcgcgttgcatttctgttctgtaaaaatgcagctcagattctttgtttgaaaaattagcgctctcgcgttgcatttttgttttacaaaaatgaagcacagattcttcgttggtaaaatagcgctttcgcgttgcatttctgttctgtaaaaatgcagctcagattctttgtttgaaaaattagcgctctcgcgttgcatttttgttctacaaaatgaagcacagatgcttcgtt

SEQ ID NO: 83 - DNA, 6481 bp, Artificial Sequence
  Synthetic construct
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatcgactacgtcgtaaggccgtttctgacagagtaaaattcttgagggaactttcaccattatgggaaatggttcaagaaggtattgacttaaactccatcaaatggtcaggtcattgagtgttttttatttgttgtatttttttttttttagagaaaatcctccaatatcaaattaggaatcgtagtttcatgattttctgttacacctaactttttgtgtggtgccctcctccttgtcaatattaatgttaaagtgcaattctttttccttatcacgttgagccattagtatcaatttgcttacctgtattcctttactatcctcctttttctccttcttgataaatgtatgtagattgcgtatatagtttcgtctaccctatgaacatattccattttgtaatttcgtgtcgtttctattatgaatttcatttataaagtttatgtacaaatatcataaaaaaagagaatctttttaagcaaggattttcttaacttcttcggcgacagcatcaccgacttcggtggtactgttggaaccacctaaatcaccagttctgatacctgcatccaaaacctttttaactgcatcttcaatggccttaccttcttcaggcaagttcaatgacaatttcaacatcattgcagcagacaagatagtggcgatagggtcaaccttattctttggcaaatctggagcagaaccgtggcatggttcgtacaaaccaaatgcggtgttcttgtctggcaaagaggccaaggacgcagatggcaacaaacccaaggaacctgggataacggaggcttcatcggagatgatatcaccaaacatgttgctggtgattataataccatttaggtgggttgggttcttaactaggatcatggcggcagaatcaatcaattgatgttgaaccttcaatgtagggaattcgttcttgatggtttcctccacagtttttctccataatcttgaagaggccaaaacattagctttatccaaggaccaaataggcaatggtggctcatgttgtagggccatgaaagcggccattcttgtgattctttgcacttctggaacggtgtattgttcactatcccaagcgacaccatcaccatcgtcttcctttctcttaccaaagtaaatacctcccactaattctctgacaacaacgaagtcagtacctttagcaaattgtggcttgattggagataagtctaaaagagagtcggatgcaaagttacatggtcttaagttggcgtacaattgaagttctttacggatttttagtaaaccttgttcaggtctaacactaccggtaccccatttaggaccacccacagcacctaacaaaacggcatcaaccttcttggaggcttccagcgcctcatctggaagtgggacacctgtagcatcgatagcagcaccaccaattaaatgattttcgaaatcgaacttgacattggaacgaacatcagaaatagctttaagaaccttaatggcttcggctgtgatttcttgaccaacgtggtcacctggcaaaacgacgatcttcttaggggcagacataggggcagacattagaatggtatatccttgaaatatatatatatattgctgaaatgtaaaaggtaagaaaagttagaaagtaagacgattgctaaccacctattggaaaaaacaataggtccttaaataatattgtcaacttcaagtattgtgatgcaagcatttagtcatgaacgcttctctattctatatgaaaagccggttccggcctctcacctttcctttttctcccaatttttcagttgaaaaaggtatatgcgtcaggcgacctctgaaattaacaaaaaatttccagtcatcgaatttgattctgtgcgatagcgcccctgtgtgttctcgttatgttgaggaaaaaaataatggttgctaagagattcgaactcttgcatcttacgatacctgagtattcccacagttaactgcggtcaagatatttcttgaatcaggcgccgcatgccggtagaggtgtggtcaataagagcgacctcatgctatacctgagaaagcaacctgacctacaggaaagagttactcaagaataagaattttcgttttaaaacctaagagtcactttaaaatttgtatacacttattttttttataacttatttaataataaaaatcataaatcataagaaattcgcttatttagaagtgtcaacaacgtatctaccaacgatttgacccttttccatcttttcgtaaatttctggcaaggtagacaagccgacaaccttgattggagacttgaccaaacctctggcgaagaagtccaaagcttctgaataagccctcgtaatatattttcatgaagaatttaggtccaaaaaaaagatgggcattaattctagtcatttaaaaaattctatagatcagaggttacatggccaagattgaaacttagaggagtatagttacataaaagaaggcaaaacgatgtataaatgaaagaaattgagatggtgcacgatgcacagttgaagtgaacttgcggggtttttcagtatctacgattcatagatctggaattgggccggcctggccactagtcatcatgatatcctcgagggccgcctgggcctgtcgagagatctctttttttgggtttggtggggtatcttcatcatcgaatagatagttatatacatcatccattgtagtggtattaaacatccctgtagtgattccaaacgcgttatacgcagtttggtccgtccaaccaggtgacagtggttttgaattattaccatcatcaattttactagccgtgatttcattattcatgaagttatcatgaacgttagaggaggcaattggttgtgaaagcgcttgagaatttgtttgagttgttatgaggttcggaccgttgctactgttagtgaaagtgaaggacaatgagctatcagcaatattcccactttgattaaaattggcggcggtacccaattcgacctttctcttcttttttggaggctcgggaattaattccgctttatccatctttgcaaagcttggagttgattgtatgcttggtatagcttgaaatattgtgcagaaaaagaaacaaggaagaaagggaacgagaacaatgacgaggaaacaaaagattaataattgcaggtctatttatacttgatagcaaagcggcaaactttttttatttcaaattcaagtaactggaaggaaggccgtataccgttgctcattagagagtagtgtgcgtgaatgaaggaaggaaaaagtttcgtgtgttcgaagatacccctcatcagctctggaacaacgacatctgttggtgctgtctttgtcgttaattttttcctttagtgtcttccatcattttttttgtcattgcggatatggtgagacaacaacgggggagagagaaaagaaaaaaaaagaaaagaagttgcatgcattcatgcaggcccggtacccagcttttgttccctttagtgagggttaattccgagcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctcggcccccctgacgagcatcacaaaaatcgacgctcaagtcagaggtggcgaaacccgacaggactataaagataccaggcgttcccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactgcccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgaaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgggtccttttcatcacgtgctataaaaataattataatttaaattttttaatataaatatataaattaaaaatagaaagtaaaaaaagaaattaaagaaaaaatagtttttgttttccgaagatgtaaaagactctagggggatcgccaacaaatactaccttttatcttgctcttcctgctctcaggtattaatgccgaattgtttcatcttgtctgtgtagaagaccacacacgaaaatcctgtgattttacattttacttatcgttaatcgaatgtatatctatttaatctgcttttcttgtctaataaatatatatgtaaagtacgctttttgttgaaattttttaaacctttgtttatttttttttcttcattccgtaactcttctaccttctttatttactttctaaaatccaaatacaaaacataaaaataaataaacacagagtaaattcccaaattattccatcattaaaagatacgaggcgcgtgtaagttacaggcaagcgatccgtcctaagaaaccattattatcatgacattaacctataaaaataggcgtatcacgaggccctttcgtc

SEQ ID NO: 84 - DNA, 6018 bp, Artificial Sequence
  Synthetic construct
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatcgactacgtcgtaaggccgtttctgacagagtaaaattcttgagggaactttcaccattatgggaaatggttcaagaaggtattgacttaaactccatcaaatggtcaggtcattgagtgttttttatttgttgtatttttttttttttagagaaaatcctccaatatcaaattaggaatcgtagtttcatgattttctgttacacctaactttttgtgtggtgccctcctccttgtcaatattaatgttaaagtgcaattctttttccttatcacgttgagccattagtatcaatttgcttacctgtattcctttactatcctcctttttctccttcttgataaatgtatgtagattgcgtatatagtttcgtctaccctatgaacatattccattttgtaatttcgtgtcgtttctattatgaatttcatttataaagtttatgtacaaatatcataaaaaaagagaatctttttaagcaaggattttcttaacttcttcggcgacagcatcaccgacttcggtggtactgttggaaccacctaaatcaccagttctgatacctgcatccaaaacctttttaactgcatcttcaatggccttaccttcttcaggcaagttcaatgacaatttcaacatcattgcagcagacaagatagtggcgatagggtcaaccttattctttggcaaatctggagcagaaccgtggcatggttcgtacaaaccaaatgcggtgttcttgtctggcaaagaggccaaggacgcagatggcaacaaacccaaggaacctgggataacggaggcttcatcggagatgatatcaccaaacatgttgctggtgattataataccatttaggtgggttgggttcttaactaggatcatggcggcagaatcaatcaattgatgttgaaccttcaatgtagggaattcgttcttgatggtttcctccacagtttttctccataatcttgaagaggccaaaacattagctttatccaaggaccaaataggcaatggtggctcatgttgtagggccatgaaagcggccattcttgtgattctttgcacttctggaacggtgtattgttcactatcccaagcgacaccatcaccatcgtcttcctttctcttaccaaagtaaatacctcccactaattctctgacaacaacgaagtcagtacctttagcaaattgtggcttgattggagataagtctaaaagagagtcggatgcaaagttacatggtcttaagttggcgtacaattgaagttctttacggatttttagtaaaccttgttcaggtctaacactaccggtaccccatttaggaccacccacagcacctaacaaaacggcatcaaccttcttggaggcttccagcgcctcatctggaagtgggacacctgtagcatcgatagcagcaccaccaattaaatgattttcgaaatcgaacttgacattggaacgaacatcagaaatagctttaagaaccttaatggcttcggctgtgatttcttgaccaacgtggtcacctggcaaaacgacgatcttcttaggggcagacataggggcagacattagaatggtatatccttgaaatatatatatatattgctgaaatgtaaaaggtaagaaaagttagaaagtaagacgattgctaaccacctattggaaaaaacaataggtccttaaataatattgtcaacttcaagtattgtgatgcaagcatttagtcatgaacgcttctctattctatatgaaaagccggttccggcctctcacctttcctttttctcccaatttttcagttgaaaaaggtatatgcgtcaggcgacctctgaaattaacaaaaaatttccagtcatcgaatttgattctgtgcgatagcgcccctgtgtgttctcgttatgttgaggaaaaaaataatggttgctaagagattcgaactcttgcatcttacgatacctgagtattcccacagttaactgcggtcaagatatttcttgaatcaggcgccttagaccgctcggccaaacaaccaattacttgttgagaaatagagtataattatcctataaatataacgtttttgaacacacatgaacaaggaagtacaggacaattgattttgaagagaatgtggattttgatgtaattgttgggattccatttttaataaggcaataatattaggtatgtggatatactagaagttctcctcgagggtcgatatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaggggggatgtgctgcaaggcgattaagttgggtaacgccagggttttcccagtcacgacgttgtaaaacgacggccagtgaattgtaatacgactcactatagggcgaattggagctccaccgcggtggcggccgctctagaactagtggatcccccgggctgcaggaattcgatatcaagcttatcgataccgtcgacctcgagggggggcccggtacccagcttttgttccctttagtgagggttaattccgagcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctcggcccccctgacgagcatcacaaaaatcgacgctcaagtcagaggtggcgaaacccgacaggactataaagataccaggcgttcccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactgcccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgaaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgggtccttttcatcacgtgctataaaaataattataatttaaattttttaatataaatatataaattaaaaatagaaagtaaaaaaagaaattaaagaaaaaatagtttttgttttccgaagatgtaaaagactctagggggatcgccaacaaatactaccttttatcttgctcttcctgctctcaggtattaatgccgaattgtttcatcttgtctgtgtagaagaccacacacgaaaatcctgtgattttacattttacttatcgttaatcgaatgtatatctatttaatctgcttttcttgtctaataaatatatatgtaaagtacgctttttgttgaaattttttaaacctttgtttatttttttttcttcattccgtaactcttctaccttctttatttactttctaaaatccaaatacaaaacataaaaataaataaacacagagtaaattcccaaattattccatcattaaaagatacgaggcgcgtgtaagttacaggcaagcgatccgtcctaagaaaccattattatcatgacattaacctataaaaataggcgtatcacgaggccctttcgtc

SEQ ID NO: 85 - DNA, 23 bp, Artificial Sequence
  Synthetic construct
cgccgcatgcattcatgcaggcc

SEQ ID NO: 86 - DNA, 17 bp, Artificial Sequence
  Synthetic construct
tgcatgaatgcatgcgg

SEQ ID NO: 87 - DNA, 5021 bp, Artificial Sequence
  Synthetic construct
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatcgactacgtcgtaaggccgtttctgacagagtaaaattcttgagggaactttcaccattatgggaaatggttcaagaaggtattgacttaaactccatcaaatggtcaggtcattgagtgttttttatttgttgtatttttttttttttagagaaaatcctccaatatcaaattaggaatcgtagtttcatgattttctgttacacctaactttttgtgtggtgccctcctccttgtcaatattaatgttaaagtgcaattctttttccttatcacgttgagccattagtatcaatttgcttacctgtattcctttactatcctcctttttctccttcttgataaatgtatgtagattgcgtatatagtttcgtctaccctatgaacatattccattttgtaatttcgtgtcgtttctattatgaatttcatttataaagtttatgtacaaatatcataaaaaaagagaatctttttaagcaaggattttcttaacttcttcggcgacagcatcaccgacttcggtggtactgttggaaccacctaaatcaccagttctgatacctgcatccaaaacctttttaactgcatcttcaatggccttaccttcttcaggcaagttcaatgacaatttcaacatcattgcagcagacaagatagtggcgatagggtcaaccttattctttggcaaatctggagcagaaccgtggcatggttcgtacaaaccaaatgcggtgttcttgtctggcaaagaggccaaggacgcagatggcaacaaacccaaggaacctgggataacggaggcttcatcggagatgatatcaccaaacatgttgctggtgattataataccatttaggtgggttgggttcttaactaggatcatggcggcagaatcaatcaattgatgttgaaccttcaatgtagggaattcgttcttgatggtttcctccacagtttttctccataatcttgaagaggccaaaacattagctttatccaaggaccaaataggcaatggtggctcatgttgtagggccatgaaagcggccattcttgtgattctttgcacttctggaacggtgtattgttcactatcccaagcgacaccatcaccatcgtcttcctttctcttaccaaagtaaatacctcccactaattctctgacaacaacgaagtcagtacctttagcaaattgtggcttgattggagataagtctaaaagagagtcggatgcaaagttacatggtcttaagttggcgtacaattgaagttctttacggatttttagtaaaccttgttcaggtctaacactaccggtaccccatttaggaccacccacagcacctaacaaaacggcatcaaccttcttggaggcttccagcgcctcatctggaagtgggacacctgtagcatcgatagcagcaccaccaattaaatgattttcgaaatcgaacttgacattggaacgaacatcagaaatagctttaagaaccttaatggcttcggctgtgatttcttgaccaacgtggtcacctggcaaaacgacgatcttcttaggggcagacataggggcagacattagaatggtatatccttgaaatatatatatatattgctgaaatgtaaaaggtaagaaaagttagaaagtaagacgattgctaaccacctattggaaaaaacaataggtccttaaataatattgtcaacttcaagtattgtgatgcaagcatttagtcatgaacgcttctctattctatatgaaaagccggttccggcctctcacctttcctttttctcccaatttttcagttgaaaaaggtatatgcgtcaggcgacctctgaaattaacaaaaaatttccagtcatcgaatttgattctgtgcgatagcgcccctgtgtgttctcgttatgttgaggaaaaaaataatggttgctaagagattcgaactcttgcatcttacgatacctgagtattcccacagttaactgcggtcaagatatttcttgaatcaggcgccgcatgcattcatgcaggcccggtacccagcttttgttccctttagtgagggttaattccgagcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctcggcccccctgacgagcatcacaaaaatcgacgctcaagtcagaggtggcgaaacccgacaggactataaagataccaggcgttcccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactgcccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgaaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgggtccttttcatcacgtgctataaaaataattataatttaaattttttaatataaatatataaattaaaaatagaaagtaaaaaaagaaattaaagaaaaaatagtttttgttttccgaagatgtaaaagactctagggggatcgccaacaaatactaccttttatcttgctcttcctgctctcaggtattaatgccgaattgtttcatcttgtctgtgtagaagaccacacacgaaaatcctgtgattttacattttacttatcgttaatcgaatgtatatctatttaatctgcttttcttgtctaataaatatatatgtaaagtacgctttttgttgaaattttttaaacctttgtttatttttttttcttcattccgtaactcttctaccttctttatttactttctaaaatccaaatacaaaacataaaaataaataaacacagagtaaattcccaaattattccatcattaaaagatacgaggcgcgtgtaagttacaggcaagcgatccgtcctaagaaaccattattatcatgacattaacctataaaaataggcgtatcacgaggccctttcgtc

SEQ ID NO: 88 - DNA, 6408 bp, Artificial Sequence
  synthetic construct
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatcgactacgtcgtaaggccgtttctgacagagtaaaattcttgagggaactttcaccattatgggaaatggttcaagaaggtattgacttaaactccatcaaatggtcaggtcattgagtgttttttatttgttgtatttttttttttttagagaaaatcctccaatatcaaattaggaatcgtagtttcatgattttctgttacacctaactttttgtgtggtgccctcctccttgtcaatattaatgttaaagtgcaattctttttccttatcacgttgagccattagtatcaatttgcttacctgtattcctttactatcctcctttttctccttcttgataaatgtatgtagattgcgtatatagtttcgtctaccctatgaacatattccattttgtaatttcgtgtcgtttctattatgaatttcatttataaagtttatgtacaaatatcataaaaaaagagaatctttttaagcaaggattttcttaacttcttcggcgacagcatcaccgacttcggtggtactgttggaaccacctaaatcaccagttctgatacctgcatccaaaacctttttaactgcatcttcaatggccttaccttcttcaggcaagttcaatgacaatttcaacatcattgcagcagacaagatagtggcgatagggtcaaccttattctttggcaaatctggagcagaaccgtggcatggttcgtacaaaccaaatgcggtgttcttgtctggcaaagaggccaaggacgcagatggcaacaaacccaaggaacctgggataacggaggcttcatcggagatgatatcaccaaacatgttgctggtgattataataccatttaggtgggttgggttcttaactaggatcatggcggcagaatcaatcaattgatgttgaaccttcaatgtagggaattcgttcttgatggtttcctccacagtttttctccataatcttgaagaggccaaaacattagctttatccaaggaccaaataggcaatggtggctcatgttgtagggccatgaaagcggccattcttgtgattctttgcacttctggaacggtgtattgttcactatcccaagcgacaccatcaccatcgtcttcctttctcttaccaaagtaaatacctcccactaattctctgacaacaacgaagtcagtacctttagcaaattgtggcttgattggagataagtctaaaagagagtcggatgcaaagttacatggtcttaagttggcgtacaattgaagttctttacggatttttagtaaaccttgttcaggtctaacactaccggtaccccatttaggaccacccacagcacctaacaaaacggcatcaaccttcttggaggcttccagcgcctcatctggaagtgggacacctgtagcatcgatagcagcaccaccaattaaatgattttcgaaatcgaacttgacattggaacgaacatcagaaatagctttaagaaccttaatggcttcggctgtgatttcttgaccaacgtggtcacctggcaaaacgacgatcttcttaggggcagacataggggcagacattagaatggtatatccttgaaatatatatatatattgctgaaatgtaaaaggtaagaaaagttagaaagtaagacgattgctaaccacctattggaaaaaacaataggtccttaaataatattgtcaacttcaagtattgtgatgcaagcatttagtcatgaacgcttctctattctatatgaaaagccggttccggcctctcacctttcctttttctcccaatttttcagttgaaaaaggtatatgcgtcaggcgacctctgaaattaacaaaaaatttccagtcatcgaatttgattctgtgcgatagcgcccctgtgtgttctcgttatgttgaggaaaaaaataatggttgctaagagattcgaactcttgcatcttacgatacctgagtattcccacagttaactgcggtcaagatatttcttgaatcaggcgccgcatgccggtagaggtgtggtcaataagagcgacctcatgctatacctgagaaagcaacctgacctacaggaaagagttactcaagaataagaattttcgttttaaaacctaagagtcactttaaaatttgtatacacttattttttttataacttatttaataataaaaatcataaatcataagaaattcgcttatttagaagtgtcaacaacgtatctaccaacgatttgacccttttccatcttttcgtaaatttctggcaaggtagacaagccgacaaccttgattggagacttgaccaaacctctggcgaagaagtccaaagcttctgaataagccctcgtaatatattttcatgaagaatttaggtccaaaaaaaagatgggcattaattctagtcatttaaaaaattctatagatcagaggttacatggccaagattgaaacttagaggagtatagttacataaaagaaggcaaaacgatgtataaatgaaagaaattgagatggtgcacgatgcacagttgaagtgaacttgcggggtttttcagtatctacgattcatagatctggaattgggccggcctggccactagtcatcatgatatcctcgagggccgcctgggcctgtcgagagatctctttttttgggtttggtggggtatcttcatcatcgaatagatagttatatacatcatccattgtagtggtattaaacatccctgtagtgattccaaacgcgttatacgcagtttggtccgtccaaccaggtgacagtggttttgaattattaccatcatcaattttactagccgtgatttcattattcatgaagttatcatgaacgttagaggaggcaattggttgtgaaagcgcttgagaatttgtttgagttgttatgaggttcggaccgttgctactgttagtgaaagtgaaggacaatgagctatcagcaatattcccactttgattaaaattggcggcggtacccaattcgacctttctcttcttttttggaggctcgggaattaattccgctttatccatctttgcagcggccgcttgcaaaagcctaggcctccaaaaaagcctcctcactacttctggaatagctcagaggcagaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagggcccggtacccagcttttgttccctttagtgagggttaattccgagcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctcggcccccctgacgagcatcacaaaaatcgacgctcaagtcagaggtggcgaaacccgacaggactataaagataccaggcgttcccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactgcccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgaaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgggtccttttcatcacgtgctataaaaataattataatttaaattttttaatataaatatataaattaaaaatagaaagtaaaaaaagaaattaaagaaaaaatagtttttgttttccgaagatgtaaaagactctagggggatcgccaacaaatactaccttttatcttgctcttcctgctctcaggtattaatgccgaattgtttcatcttgtctgtgtagaagaccacacacgaaaatcctgtgattttacattttacttatcgttaatcgaatgtatatctatttaatctgcttttcttgtctaataaatatatatgtaaagtacgctttttgttgaaattttttaaacctttgtttatttttttttcttcattccgtaactcttctaccttctttatttactttctaaaatccaaatacaaaacataaaaataaataaacacagagtaaattcccaaattattccatcattaaaagatacgaggcgcgtgtaagttacaggcaagcgatccgtcctaagaaaccattattatcatgacattaacctataaaaataggcgtatcacgaggccctttcgtc

SEQ ID NO: 89 - DNA, 6308 bp, Artificial Sequence
  synthetic construct
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatcgactacgtcgtaaggccgtttctgacagagtaaaattcttgagggaactttcaccattatgggaaatggttcaagaaggtattgacttaaactccatcaaatggtcaggtcattgagtgttttttatttgttgtatttttttttttttagagaaaatcctccaatatcaaattaggaatcgtagtttcatgattttctgttacacctaactttttgtgtggtgccctcctccttgtcaatattaatgttaaagtgcaattctttttccttatcacgttgagccattagtatcaatttgcttacctgtattcctttactatcctcctttttctccttcttgataaatgtatgtagattgcgtatatagtttcgtctaccctatgaacatattccattttgtaatttcgtgtcgtttctattatgaatttcatttataaagtttatgtacaaatatcataaaaaaagagaatctttttaagcaaggattttcttaacttcttcggcgacagcatcaccgacttcggtggtactgttggaaccacctaaatcaccagttctgatacctgcatccaaaacctttttaactgcatcttcaatggccttaccttcttcaggcaagttcaatgacaatttcaacatcattgcagcagacaagatagtggcgatagggtcaaccttattctttggcaaatctggagcagaaccgtggcatggttcgtacaaaccaaatgcggtgttcttgtctggcaaagaggccaaggacgcagatggcaacaaacccaaggaacctgggataacggaggcttcatcggagatgatatcaccaaacatgttgctggtgattataataccatttaggtgggttgggttcttaactaggatcatggcggcagaatcaatcaattgatgttgaaccttcaatgtagggaattcgttcttgatggtttcctccacagtttttctccataatcttgaagaggccaaaacattagctttatccaaggaccaaataggcaatggtggctcatgttgtagggccatgaaagcggccattcttgtgattctttgcacttctggaacggtgtattgttcactatcccaagcgacaccatcaccatcgtcttcctttctcttaccaaagtaaatacctcccactaattctctgacaacaacgaagtcagtacctttagcaaattgtggcttgattggagataagtctaaaagagagtcggatgcaaagttacatggtcttaagttggcgtacaattgaagttctttacggatttttagtaaaccttgttcaggtctaacactaccggtaccccatttaggaccacccacagcacctaacaaaacggcatcaaccttcttggaggcttccagcgcctcatctggaagtgggacacctgtagcatcgatagcagcaccaccaattaaatgattttcgaaatcgaacttgacattggaacgaacatcagaaatagctttaagaaccttaatggcttcggctgtgatttcttgaccaacgtggtcacctggcaaaacgacgatcttcttaggggcagacataggggcagacattagaatggtatatccttgaaatatatatatatattgctgaaatgtaaaaggtaagaaaagttagaaagtaagacgattgctaaccacctattggaaaaaacaataggtccttaaataatattgtcaacttcaagtattgtgatgcaagcatttagtcatgaacgcttctctattctatatgaaaagccggttccggcctctcacctttcctttttctcccaatttttcagttgaaaaaggtatatgcgtcaggcgacctctgaaattaacaaaaaatttccagtcatcgaatttgattctgtgcgatagcgcccctgtgtgttctcgttatgttgaggaaaaaaataatggttgctaagagattcgaactcttgcatcttacgatacctgagtattcccacagttaactgcggtcaagatatttcttgaatcaggcgccgcatgccggtagaggtgtggtcaataagagcgacctcatgctatacctgagaaagcaacctgacctacaggaaagagttactcaagaataagaattttcgttttaaaacctaagagtcactttaaaatttgtatacacttattttttttataacttatttaataataaaaatcataaatcataagaaattcgcttatttagaagtgtcaacaacgtatctaccaacgatttgacccttttccatcttttcgtaaatttctggcaaggtagacaagccgacaaccttgattggagacttgaccaaacctctggcgaagaagtccaaagcttctgaataagccctcgtaatatattttcatgaagaatttaggtccaaaaaaaagatgggcattaattctagtcatttaaaaaattctatagatcagaggttacatggccaagattgaaacttagaggagtatagttacataaaagaaggcaaaacgatgtataaatgaaagaaattgagatggtgcacgatgcacagttgaagtgaacttgcggggtttttcagtatctacgattcatagatctggaattgggccggcctggccactagtcatcatgatatcctcgagggccgcctgggcctgtcgagagatctctttttttgggtttggtggggtatcttcatcatcgaatagatagttatatacatcatccattgtagtggtattaaacatccctgtagtgattccaaacgcgttatacgcagtttggtccgtccaaccaggtgacagtggttttgaattattaccatcatcaattttactagccgtgatttcattattcatgaagttatcatgaacgttagaggaggcaattggttgtgaaagcgcttgagaatttgtttgagttgttatgaggttcggaccgttgctactgttagtgaaagtgaaggacaatgagctatcagcaatattcccactttgattaaaattggcggcggtacccaattcgacctttctcttcttttttggaggctcgggaattaattccgctttatccatctttgcagcggccgcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagggcccggtacccagcttttgttccctttagtgagggttaattccgagcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctcggcccccctgacgagcatcacaaaaatcgacgctcaagtcagaggtggcgaaacccgacaggactataaagataccaggcgttcccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactgcccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgaaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgggtccttttcatcacgtgctataaaaataattataatttaaattttttaatataaatatataaattaaaaatagaaagtaaaaaaagaaattaaagaaaaaatagtttttgttttccgaagatgtaaaagactctagggggatcgccaacaaatactaccttttatcttgctcttcctgctctcaggtattaatgccgaattgtttcatcttgtctgtgtagaagaccacacacgaaaatcctgtgattttacattttacttatcgttaatcgaatgtatatctatttaatctgcttttcttgtctaataaatatatatgtaaagtacgctttttgttgaaattttttaaacctttgtttatttttttttcttcattccgtaactcttctaccttctttatttactttctaaaatccaaatacaaaacataaaaataaataaacacagagtaaattcccaaattattccatcattaaaagatacgaggcgcgtgtaagttacaggcaagcgatccgtcctaagaaaccattattatcatgacattaacctataaaaataggcgtatcacgaggccctttcgtc

SEQ ID NO: 90 - DNA, 8068 bp, Artificial Sequence
  synthetic construct
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgtttcgaagatatcgttgacattgattattgtctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcacttaagctggagctttgggaggagacggggaggacagactggaggcgtgggcccactagtgtttagtgaaccgtcagatcgcctggagacgccatccacgctgttttgacctccatagaagacaccgggaccgatccagcctccggactctagcctcgagcccaagcttggtaccgagctcggatccagccaccatgggagtcaaagttctgtttgccctgatctgcatcgctgnggccgaggccaagcccaccgagaacaacgaagacttcaacatcgtggccgtggccagcaacttcgcgaccacggatctcgatgctgaccgcgggaagttgcccggcaagaagctgccgctggaggtgctcaaagagctggaagccaatgcccggaaagctggctgcaccaggggctgtctgatctgcctgtcccacatcaagtgcacgcccaagatgaagaagttcatcccaggacgctgccacacctacgaaggcgacaaagagtccgcacagggcggcataggcgaggcgatcgtcgacattcctgagattcctgggttcaaggacttggagcccctggagcagttcatcgcacaggtcgatctgtgtgtggactgcacaactggctgcctcaaagggcttgccaacgtgcagtgttctgacctgctcaagaagtggctgccgcaacgctgtgcgacctttgccagcaagatccagggccaggtggacaagatcaagggggccggtggtgactaagcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtacaaccggtctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattttaattaacgttggcaccatgctgctgctgctgctgctgctgggcctgaggctacagctctccctgggcatcatcccagttgaggaggagaacccggacttctggaaccgcgaggcagccgaggccctgggtgccgccaagaagctgcagcctgcacagacagccgccaagaacctcatcatcttcctgggcgatgggatgggggtgtctacggtgacagctgccaggatcctaaaagggcagaagaaggacaaactggggcctgagatacccctggccatggaccgcttcccatatgtggctctgtccaagacatacaatgtagacaaacatgtgccagacagtggagccacagccacggcctacctgtgcggggtcaagggcaacttccagaccattggcttgagtgcagccgcccgctttaaccagtgcaacacgacacgcggcaacgaggtcatctccgtgatgaatcgggccaagaaagcagggaagtcagtgggagtggtaaccaccacacgagtgcagcacgcctcgccagccggcacctacgcccacacggtgaaccgcaactggtactcggacgccgacgtgcctgcctcggcccgccaggaggggtgccaggacatcgctacgcagctcatctccaacatggacattgacgtgatcctaggtggaggccgaaagtacatgtttcgcatgggaaccccagaccctgagtacccagatgactacagccaaggtgggaccaggctggacgggaagaatctggtgcaggaatggctggcgaagcgccagggtgcccggtatgtgtggaaccgcactgagctcatgcaggcttccctggacccgtctgtgacccatctcatgggtctctttgagcctggagacatgaaatacgagatccaccgagactccacactggacccctccctgatggagatgacagaggctgccctgcgcctgctgagcaggaacccccgcggcttcttcctcttcgtggagggtggtcgcatcgaccatggtcatcatgaaagcagggcttaccgggcactgactgagacgatcatgttcgacgacgccattgagagggcgggccagctcaccagcgaggaggacacgctgagcctcgtcactgccgaccactcccacgtcttctccttcggaggctaccccctgcgagggagctccatcttcgggctggcccctggcaaggcccgggacaggaaggcctacacggtcctcctatacggaaacggtccaggctatgtgctcaaggacggcgcccggccggatgttaccgagagcgagagcgggagccccgagtatcggcagcagtcagcagtgcccctggacgaagagacccacgcaggcgaggacgtggcggtgttcgcgcgcggcccgcaggcgcacctggttcacggcgtgcaggagcagaccttcatagcgcacgtcatggccttcgccgcctgcctggagccctacaccgcctgcgacctggcgccccccgccggcaccaccgacgccgcgcacccgggttactctagagtcggggcggccggctaggtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtacctagaagttcctattccgaagttcctattctctagaaagtataggaacttccttggccaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagcacgtactacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 91 - DNA, 54 bp, Artificial Sequence
  synthetic construct
ttaagtggtttaggaaagcaggagctattcaggaagcaggggtcctcaccggta

SEQ ID NO: 92 - DNA, 54 bp, Artificial Sequence
  synthetic construct
ctagtaccggtgaggacccctgcttcctgaatagctcctgctttcctaaaccac

SEQ ID NO: 93 - DNA, 6083 bp, Artificial Sequence
  Synthetic construct
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacgggccctctagagatatcatggatgctaagtccctgacagcgtggagccgcacactggttaccttcaaagatgttttcgtggatttcacccgcgaagagtggaaactgctggataccgcacagcagattgtgtatcgcaacgttatgctggaaaactacaagaatctggttagcctgggctatcagctgacaaaacccgacgtcatcctgcgtctggaaaagggtgaagagccgtggctggttgaacgggagattcaccaggagacacatcctgattctgaaactgcctttgagatcaaaagctccgtcagtccgaaaaagaaacgtaaagtggggctcgagcccggggaaaagccatataaatgccccgagtgcggcaaatcattcagccaaagtagcaacttagtaagacaccagcgcacccataccggggaaaagccatataaatgccccgagtgcggcaaatcattcagccaaagtagcaacttagtaagacaccagcgcacccataccggggaaaagccatataaatgccccgagtgcggcaaatcattcagccaaagtagcaacttagtaagacaccagcgcacccataccggtgagcagaaactcatctctgaagaagatctggaacaaaagttgatttcagaagaagatctggaacagaagctcatctctgaggaagatctgtaagcggccgcgaattccaccacactggactagtggatccgagctcggtaccaagcttaagtttaaaccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 94 - DNA, 5916 bp, Artificial Sequence
  Synthetic construct
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacgggccctctagagatatcatgccgaaaaagaaacgtaaagtggggctcgagcccggggaaaagccatataaatgccccgagtgcggcaaatcattcagccaaagtagcaacttagtaagacaccagcgcacccataccggggaaaagccatataaatgccccgagtgcggcaaatcattcagccaaagtagcaacttagtaagacaccagcgcacccataccggggaaaagccatataaatgccccgagtgcggcaaatcattcagccaaagtagcaacttagtaagacaccagcgcacccataccggtggcggcagcggcggcagcgaattccgcacactggttaccttcaaagatgttttcgtggatttcacccgcgaagagtggaaactgctggataccgcacagcagattgtgtatcgcaacgttatgctggaaaactacaagaatctggttagcctgggctatggatccgagcagaaactcatctctgaagaagatctggaacaaaagttgatttcagaagaagatctggaacagaagctcatctctgaggaagatctgtaagcggccgcaagcttaagtttaaaccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 95 - DNA, 5897 bp, Artificial Sequence
  Synthetic construct
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacgggccctctagagatatcatggcggcggcggttcggatgaacatccagatgctgctggaggcggccgactatctggagcggcgggagagagaagctgaacatggttatgcctccatgttaccatacccgaaaaagaaacgtaaagtggggctcgagcccggggaaaagccatataaatgccccgagtgcggcaaatcattcagccaaagtagcaacttagtaagacaccagcgcacccataccggggaaaagccatataaatgccccgagtgcggcaaatcattcagccaaagtagcaacttagtaagacaccagcgcacccataccggggaaaagccatataaatgccccgagtgcggcaaatcattcagccaaagtagcaacttagtaagacaccagcgcacccataccggtgagcagaaactcatctctgaagaagatctggaacaaaagttgatttcagaagaagatctggaacagaagctcatctctgaggaagatctgtaagcggccgcgaattccaccacactggactagtggatccgagctcggtaccaagcttaagtttaaaccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 96 - DNA, 6198 bp, Artificial Sequence
  Synthetic construct
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacgggccctctagagatatcatgccgaaaaagaaacgtaaagtggggctcgagcccggggaaaagccctacaagtgccctgagtgtgggaagtccttttcttcaagacgcacgtgccgcgctcaccagcggacacataccggggagaagccctataaatgtccagaatgtggaaagtcctttagcacgtcagggaacttagtaagacaccagcgaactcataccggggagaagccatataaatgtcccgagtgtggcaagtccttttctagatcagataatttagtaagacatcagagaacgcacaccggggaaaagccctacaagtgcccggaatgcggcaagtcttttagcaccagcggacatttagtaagacaccagagaacccacaccggggaaaaaccctataaatgccccgagtgtggtaagtcattctctcaaagcggggatttaagaagacaccagagaacccacaccggggaaaaaccgtataaatgtcctgagtgcggtaagtctttttccgactgtagagacttagcgagacaccaacgtactcataccggtggcggcagcggcggcagcgaattcgggcgcgccgacgcgctggacgatttcgatctcgacatgctgggttctgatgccctcgatgactttgacctggatatgttgggaagcgacgcattggatgactttgatctggacatgctcggctccgatgctctggacgatttcgatctcgatatgttaattaacggatccgagcagaaactcatctctgaagaagatctggaacaaaagttgatttcagaagaagatctggaacagaagctcatctctgaggaagatctgtaagcggccgcaagcttaagtttaaaccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 97 - DNA, 10723 bp, Artificial Sequence
  synthetic construct
actcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcctcgttcattcacgtttttgaacccgtggaggacgggcagactcgcggtgcaaatgtgttttacagcgtgatggagcagatgaagatgctcgacacgctgcagaacacgcagctagattaaccctagaaagataatcatattgtgacgtacgttaaagataatcatgcgtaaaattgacgcatgtgttttatcggtctgtatatcgaggtttatttattaatttgaatagatattaagttttattatatttacacttacatactaataataaattcaacaaacaatttatttatgtttatttatttattaaaaaaaaacaaaaactcaaaatttcttctataaagtaacaaaacttttatgagggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaaggcctccaaggcctactagtaacggccgccagtgtgctggaattcgcccttggtacctgctttctctgaccagcattctctcccctgggcctgtgccgctttctgtctgcagcttgtggcctgggtcacctctacggctggcccagatccttccctgccgcctccttcaggttccgtcttcctccactccctcttccccttgctctctgctgtgttgctgcccaaggatgctctttccggagcacttccttctcggcgctgcaccacgtgatgtcctctgagcggatcctccccgtgtctgggtcctctccgggcatctctcctccctcacccaaccccatgccgtcttcactcgctgggttcccttttccttctccttctggggcctgtgccatctctcgtttcttaggatggccttctccgacggatgtctcccttgcgtcccgcctccccttcttgtaggcctgcatcatcaccgtttttctggacaaccccaaagtaccccgtctccctggctttagccacctctccatcctcttgctttctttgcctggacaccccgttctcctgtggattcgggtcacctctcactcctttcatttgggcagctcccctaccccccttacctctctagtctgtgctagctcttccagccccctgtcatggcatcttccaggggtccgagagctcagctagtcttcttcctccaacccgggcccctatgtccacttcaggacagcatgtttgctgcctccagggatcctgtgtccccgagctgggaccaccttatattcccagggccggttaatgtggctctggttctgggtacttttatctgtcccctccaccccacagtggggcacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctccctatcagtgatagagatctccctatcagtgatagagatcgtcgacgagctcgtttagtgaaccgtcagatcgcctggagacgccatccacgctgttttgacctccatagaagacaccgggaccgatccagcctccggactctagcgtttaaacgatatcatggcggcggcggttcggatgaacatccagatgctgctggaggcggccgactatctggagcggcgggagagagaagctgaacatggttatgcctccatgttaccatacccgaaaaagaaacgtaaagtggggctcgagcccggggagaagccatataaatctcccgagtccggcaagtccttttctagatcagataatttagtaagacatcagagaacgcacaccggggagaagccgtacaagagccctgaatctggtaagtcattttcgagaagtgatgaattagtaagacaccagcggactcataccggggaaaagccctacaagagcccggaaagcggcaagtcttttagcaccagcggacatttagtaagacaccagagaacccacaccggggagaagccttataagtcccctgagagcggcaaaagcttcagcgatcctggaaatttagtaagacaccaacgcacccacaccggggaaaaaccttacaagtctcctgagagcggcaagagcttctctcaatcaagttcattagtaagacaccagaggactcataccggggagaaaccatacaagtccccagagagcgggaaaaactttagtacaagcggtgagttagtaagacaccaacgaacacacaccggtggatccggcggcagcggcggcagcgtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaagcggccgcttcgaatttaaatcggatccctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggagatctgcggccgcgaaggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttgtgagtttggggacccttgattgttctttctttttcgctattgtaaaattcatgttatatggagggggcaaagttttcagggtgttgtttagaatgggaagatgtcccttgtatcaccatggaccctcatgataattttgtttctttcactttctactctgttgacaaccattgtctcctcttattttcttttcattttctgtaactttttcgttaaactttagcttgcatttgtaacgaatttttaaattcacttttgtttatttgtcagattgtaagtactttctctaatcacttttttttcaaggcaatcagggtatattatattgtacttcagcacagttttagagaacaattgttataattaaatgataaggtagaatatttctgcatataaattctggctggcgtggaaatattcttattggtagaaacaactacatcctggtcatcatcctgcctttctctttatggttacaatgatatacactgtttgagatgaggataaaatactctgagtccaaaccgggcccctctgctaaccatgttcatgccttcttctttttcctacagctcctgggcaacgtgctggttattgtgctgtctcatcattttggcaaagaattgtaatacgactcactatagggcgaattgatatgtctagattagataaaagtaaagtgattaacagcgcattagagctgcttaatgaggtcggaatcgaaggtttaacaacccgtaaactcgcccagaagctaggtgtagagcagcctacattgtattggcatgtaaaaaataagcgggctttgctcgacgccttagccattgagatgttagataggcaccatactcacttttgccctttagaaggggaaagctggcaagattttttacgtaataacgctaaaagttttagatgtgctttactaagtcatcgcgatggagcaaaagtacatttaggtacacggcctacagaaaaacagtatgaaactctcgaaaatcaattagcctttttatgccaacaaggtttttcactagagaatgcattatatgcactcagcgctgtggggcattttactttaggttgcgtattggaagatcaagagcatcaagtcgctaaagaagaaagggaaacacctactactgatagtatgccgccattattacgacaagctatcgaattatttgatcaccaaggtgcagagccagccttcttattcggccttgaattgatcatatgcggattagaaaaacaacttaaatgtgaaagtgggtccgcgtacagcggatcccgggaattcagatcttatgcgatcgagggcagaggaagtcttctaacatgcggtgacgtggaggagaatcccggccctatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgaaatcaacctctggattacaaaatttgtgaaagattgactggtattcttaactatgttgctccttttacgctatgtggatacgctgctttaatgcctttgtatcagttaacttgtttattgcagcttataatggttacaaataaagcaatagcatcacaaatttcacaaataaagcatttttttcactgcattctagttgtggtttgtccaaactcatcaatgtatcttatcatgtctggaattgactcaaatgatgtcaattagtctatcagaagctatctggtctcccttccgggggacaagacatccctgtttaatatttaaacagcagtgttcccaaactgggttcttatatcccttgctctggtcaaccaggttgcagggtttcctgtcctcacaggaacgaagtccctaaagaaacagtggcagccaggtttagccccggaattgactggattccttttttagggcccattggtatggtgtacactactagggacaggattggtgacagaaaagccccatccttaggcctcctccttcctagtctcctgatattgggtctaacccccacctcctgttaggcagattccttatctggtgacacacccccatttcctggagccatctctctccttgccagaacctctaaggtttgcttacgatggagccagagaggatcctgggagggagagcttggcagggggtgggagggaagggggggatgcgtgacctgcccggttctcagtggccaccctgcgctaccctctcccagaacctgagctgctctgacgcggctgtctggtgcgtttcactgatcctggtgctgcagcttccttacacttcccaagaggagaagcagtttggaaaaacaaaatcagaataagttggtcctgagttctaactttggctcttcacctttctagtccccaatttatattgttcctccgtgcgtcagttttacctgtgagataaggccagtagccagccccgtcctggcagggctgtggtgaggaggggggtgtccgtgtggaaaactccctttgtgagaatggtgcgtcctaggtgttcaccaggtcgtggccgcctctactccctttctctttctccatccttctttccttaaagagtccccagtgctatctgggacatattcctccgcccagagcagggtcccgcttccctaaggccctgctctgggcttctgggtttgagtccttggcaagcccaggagaggcgctcaggcttccctgtcccccttcctcgtccaccatctcatgcccctggctctcctgccccttccctacaggggttcctggctctgctctctcgagatgcatgcgtcaattttacgcagactatctttctagggttaatctagctgcatcaggatcatatcgtcgggtcttttttccggctcagtcatcgcccaagctggcgctatctgggcatcggggaggaagaagcccgtgccttttcccgcgaggttgaagcggcatggaaagagtttgccgaggatgactgctgctgcattgacgttgagcgaaaacgcacgtttaccatgatgattcgggaaggtgtggccatgcacgcctttaacggtgaactgttcgttcaggccacctgggataccagttcgtcgcggcttttccggacacagttccggatggtcagcccgaagcgcatcagcaacccgaacaataccggcgacagccggaactgccgtgccggtgtgcagattaatgacagcggtgcggcgctgggatattacgtcagcgaggacgggtatcctggctggatgccgcagaaatggacatggataccccgtgagttacccggcgggcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcat

SEQ ID NO: 98 - DNA, 5185 bp, Artificial Sequence
  Synthetic construct
tggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgctagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatcgatctcgatcccgcgaaattaatacgactcactataggggaattgtgagcggataacaattcccctctagaaataattttgtttaactttaagaaggagatatacatatgcaccaccaccaccaccacggctatggccgcaaaaaacgccgccagcgccgccgcggctatccgtatgatgagccggattatgccccatgggatatcggggatccgaattctgtacaggccttggcgcgcctgcaggcgagctccgtcgacaagcttgcggccgcactcgagcaccaccaccaccaccaccaccactaattgattaatacctaggctgctaaacaaagcccgaaaggaagctgagttggctgctgccaccgctgagcaataactagcataaccccttggggcctctaaacgggtcttgaggggttttttgctgaaaggaggaactatatccggat

SEQ ID NO: 99 - DNA, 5866 bp, Artificial Sequence
  synthetic construct
tggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgctagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatcgatctcgatcccgcgaaattaatacgactcactataggggaattgtgagcggataacaattcccctctagaaataattttgtttaactttaagaaggagatatacatatgcaccaccaccaccaccacggctatggccgcaaaaaacgccgccagcgccgccgcggctatccgtatgatgtgccggattatgccccatgggatatcatggcggcggcggttcggatgaacatccagatgctgctggaggcggccgactatctggagcggcgggagagagaagctgaacatggttatgcctccatgttaccatacccgaaaaagaaacgtaaagtggggctcgagcccggggagaaaccttataaatgcccagaatgcgggaaatcgttcagtcaaagagcacatttagaaagacatcaacggacccacaccggggagaagccatacaagtgccctgaatgtggcaagtccttttcaagagccgataacctgacagaacaccaaaggacgcataccggggaaaaaccttataagtgtcccgagtgcggcaagagtttcagtcacaaaaacgcacttcagaatcatcagaggacacataccggggagaagccctataaatgtccagaatgtggaaagtcctttagcacgtcagggaacttagtaagacaccagcgaactcataccggggagaaaccttataaatgcccagaatgcgggaaatcgttcagtcaaagagcacatttagaaagacatcaacggacccacaccggggaaaaaccttacaagtgccctgagtgcggcaagagcttctctcaatcaagttcattagtaagacaccagaggactcataccggtgagcagaaactcatctctgaagaagatctggaacaaaagttgatttcagaagaagatctggaacagaagctcatctctgaggaagatctgtaagcggccgcactcgagcaccaccaccaccaccaccaccactaattgattaatacctaggctgctaaacaaagcccgaaaggaagctgagttggctgctgccaccgctgagcaataactagcataaccccttggggcctctaaacgggtcttgaggggttttttgctgaaaggaggaactatatccggat

SEQ ID NO: 100 - DNA, 5866 bp, Artificial Sequence
  synthetic construct
tggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgctagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatcgatctcgatcccgcgaaattaatacgactcactataggggaattgtgagcggataacaattcccctctagaaataattttgtttaactttaagaaggagatatacatatgcaccaccaccaccaccacggctatggccgcaaaaaacgccgccagcgccgccgcggctatccgtatgatgtgccggattatgccccatgggatatcatggcggcggcggttcggatgaacatccagatgctgctggaggcggccgactatctggagcggcgggagagagaagctgaacatggttatgcctccatgttaccatacccgaaaaagaaacgtaaagtggggctcgagcccggggagaagccctataaatgtccagaatgtggaaagtcctttagcacgtcagggaacttagtaagacaccagcgaactcataccggggagaagccatacaagtgccctgaatgtggcaagtccttttcaagagccgataacctgacagaacaccaaaggacgcataccggggaaaaaccttataagtgtcccgagtgcggcaagagtttcagtcacaaaaacgcacttcagaatcatcagaggacacataccggggaaaagccctacaagtgtcctgagtgcggaaagtctttctccactagcggttcattagtaagacaccagaggacacacaccggggagaaaccttataaatgcccagaatgcgggaaatcgttcagtcaaagagcacatttagaaagacatcaacggacccacaccggggagaaaccatacaaatgccccgagtgtggaaagtcatttagtgatccaggcgcattagtaagacatcagcggacacataccggtgagcagaaactcatctctgaagaagatctggaacaaaagttgatttcagaagaagatctggaacagaagctcatctctgaggaagatctgtaagcggccgcactcgagcaccaccaccaccaccaccaccactaattgattaatacctaggctgctaaacaaagcccgaaaggaagctgagttggctgctgccaccgctgagcaataactagcataaccccttggggcctctaaacgggtcttgaggggttttttgctgaaaggaggaactatatccggat

SEQ ID NO: 101 - DNA, 5866 bp, Artificial Sequence
  synthetic construct
tggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgctagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatcgatctcgatcccgcgaaattaatacgactcactataggggaattgtgagcggataacaattcccctctagaaataattttgtttaactttaagaaggagatatacatatgcaccaccaccaccaccacggctatggccgcaaaaaacgccgccagcgccgccgcggctatccgtatgatgtgccggattatgccccatgggatatcatggcggcggcggttcggatgaacatccagatgctgctggaggcggccgactatctggagcggcgggagagagaagctgaacatggttatgcctccatgttaccatacccgaaaaagaaacgtaaagtggggctcgagcccggggagaagccctataaatgtccagaatgtggaaagtcctttagcacgtcagggaacttagtaagacaccagcgaactcataccggggagaagccatacaagtgccctgaatgtggcaagtccttttcaagagccgataacctgacagaacaccaaaggacgcataccggggaaaaaccttataagtgtcccgagtgcggcaagagtttcagtcacaaaaacgcacttcagaatcatcagaggacacataccggggaaaaaccctataaatgccccgagtgtggtaagtcattctctcaaagcggggatttaagaagacaccagagaacccacaccggggagaaaccttataaatgcccagaatgcgggaaatcgttcagtcaaagagcacatttagaaagacatcaacggacccacaccggggaaaaaccttacaagtgccctgagtgcggcaagagcttctctcaatcaagttcattagtaagacaccagaggactcataccggtgagcagaaactcatctctgaagaagatctggaacaaaagttgatttcagaagaagatctggaacagaagctcatctctgaggaagatctgtaagcggccgcactcgagcaccaccaccaccaccaccaccactaattgattaatacctaggctgctaaacaaagcccgaaaggaagctgagttggctgctgccaccgctgagcaataactagcataaccccttggggcctctaaacgggtcttgaggggttttttgctgaaaggaggaactatatccggat

SEQ ID NO: 102 - DNA, 5866 bp, Artificial Sequence
  synthetic construct
tggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgctagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatcgatctcgatcccgcgaaattaatacgactcactataggggaattgtgagcggataacaattcccctctagaaataattttgtttaactttaagaaggagatatacatatgcaccaccaccaccaccacggctatggccgcaaaaaacgccgccagcgccgccgcggctatccgtatgatgtgccggattatgccccatgggatatcatggcggcggcggttcggatgaacatccagatgctgctggaggcggccgactatctggagcggcgggagagagaagctgaacatggttatgcctccatgttaccatacccgaaaaagaaacgtaaagtggggctcgagcccggggagaaaccttataaatgcccagaatgcgggaaatcgttcagtcaaagagcacatttagaaagacatcaacggacccacaccggggagaaaccatacaagtgtccagagtgcgggaaaagctttagtacaagcggtgagttagtaagacaccaacgaacacacaccggggagaaaccatacaagtgtccagagtgcgggaaaagctttagtacaagcggtgagttagtaagacaccaacgaacacacaccggggagaaaccatacaaatgccccgagtgtggaaagtcatttagtgatccaggcgcattagtaagacatcagcggacacataccggggagaaaccatacaaatgccccgagtgtggaaagtcatttagtgatccaggcgcattagtaagacatcagcggacacataccggggaaaagccctataagtgtcccgaatgcggcaagagttttagtactactggcgcactcacagaacaccagcgcactcacaccggtgagcagaaactcatctctgaagaagatctggaacaaaagttgatttcagaagaagatctggaacagaagctcatctctgaggaagatctgtaagcggccgcactcgagcaccaccaccaccaccaccaccactaattgattaatacctaggctgctaaacaaagcccgaaaggaagctgagttggctgctgccaccgctgagcaataactagcataaccccttggggcctctaaacgggtcttgaggggttttttgctgaaaggaggaactatatccggat

SEQ ID NO: 103 - DNA, 5866 bp, Artificial Sequence
  synthetic construct
tggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgctagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatcgatctcgatcccgcgaaattaatacgactcactataggggaattgtgagcggataacaattcccctctagaaataattttgtttaactttaagaaggagatatacatatgcaccaccaccaccaccacggctatggccgcaaaaaacgccgccagcgccgccgcggctatccgtatgatgtgccggattatgccccatgggatatcatggcggcggcggttcggatgaacatccagatgctgctggaggcggccgactatctggagcggcgggagagagaagctgaacatggttatgcctccatgttaccatacccgaaaaagaaacgtaaagtggggctcgagcccggggagaagccctacaagtgtccagaatgcggaaagagtttctccagaagtgacaaattagtaagacaccagagaacccataccggggagaagccgtacaagtgccctgaatgtggtaagtcattttcgagaagtgatgaattagtaagacaccagcggactcataccggggaaaaaccgtacaagtgtcctgagtgcgggaagagtttctccgatccgggccacttagtaagacatcagaggacacataccggggagaagccatataaatgtcccgagtgtggcaagtccttttctagatcagataatttagtaagacatcagagaacgcacaccggggagaagccatacaagtgtcccgaatgcgggaagtcattctccagaagtgacgatttagtaagacatcagcgcacgcacaccggggagaaaccctataagtgtcccgaatgcgggaaatcattctctcatacagggcatctgctcgaacatcaaaggacgcacaccggtgagcagaaactcatctctgaagaagatctggaacaaaagttgatttcagaagaagatctggaacagaagctcatctctgaggaagatctgtaagcggccgcactcgagcaccaccaccaccaccaccaccactaattgattaatacctaggctgctaaacaaagcccgaaaggaagctgagttggctgctgccaccgctgagcaataactagcataaccccttggggcctctaaacgggtcttgaggggttttttgctgaaaggaggaactatatccggat

(19) United States
(12) Patent Application Publication
(10) Pub. No.: US 2016/0046681 A1
(43) Pub. Date: Feb. 18, 2016

(54) ARTIFICIAL TRANSCRIPTION FACTORS REGULATING NUCLEAR RECEPTORS AND THEIR THERAPEUTIC USE

(71) Applicant: ALIOPHTHA AG, Basel (CH)

(72) Inventors: Albert NEUTZNER, Schliengen (DE); Josef FLAMMER, Binningen (CH); Alice HUXLEY, Binningen (CH)

(73) Assignee: ALIOPHTHA AG, Basel (CH)

(21) Appl. No.: 14/781,688

(22) PCT Filed: Apr. 2, 2014
(86) PCT No.: PCT/EP2014/056594

§ 371 Date: Oct. 1, 2015

(30) Foreign Application Priority Data
Apr. 3, 2013 (EP) 13162198.9

(51) Int. Cl. C07K 14/47

(52) U.S. Cl. CPC C07K 14/4702; C07K 2319/81

(57) ABSTRACT

The invention relates to an artificial transcription factor comprising a polydactyl zinc finger protein targeting specifically a promoter region of a nuclear receptor genefused to an inhibitory or activatory protein domain, a nuclear localization sequence, and a protein transduction domain. In particular examples these promoter regions of a nuclear receptor gene regulate the expression of the glucocorticoid receptor, the androgen receptor, or the estrogen receptor ESR1. Artificial transcription factors directed against the glucocorticoid receptor are useful in the treatment of diseases modulated by glucocorticoids, such as inflammatory processes, diabetes, obesity, coronary artery disease, asthma, celiac disease and lupus erythematosus. Artificial transcription factors directed against the androgen receptor are useful in the treatment of diseases modulated by testosterone, such as various cancers, coronary artery disease, metabolic disorders such as obesity or diabetes or mood disorders such as schizophrenia, depression or attention deficit hyperactivity disorder. Artificial transcription factors directed against the estrogen receptor are useful in the treatment of diseases modulated by estrogens, such as various cancers, cardiovascular disease, osteoporosis or mood disorders.

### FIELD OF THE INVENTION

The invention relates to artificial transcription factors comprising a polydactyl zinc finger protein targeting specifically a promoter region of a nuclear receptor gene fused to an inhibitory or activatory domain, a nuclear localization sequence, and a protein transduction domain, and their use in treating diseases caused or modulated by the activity of such nuclear receptors.

### BACKGROUND OF THE INVENTION

Artificial transcription factors (ATFs) are proposed to be useful tools for modulating gene expression (Sera T., 2009, Adv Drug Deliv Rev 61, 513-526). Many naturally occurring transcription factors, influencing expression either through repression or activation of gene transcription, possess complex specific domains for the recognition of a certain DNA sequence. This makes them unattractive targets for manipulation if one intends to modify their specificity and target gene(s). However, a certain class of transcription factors contains several so called zinc finger (ZF) domains, which are modular and therefore lend themselves to genetic engineering. Zinc fingers are short (30 amino acids) DNA binding motifs targeting almost independently three DNA base pairs. A protein containing several such zinc fingers fused together is thus able to recognize longer DNA sequences. A hexameric zinc finger protein (ZFP) recognizes an 18 base pairs (bp) DNA target, which is almost unique in the entire human genome. Initially thought to be completely context independent, more in-depth analyses revealed some context specificity for zinc fingers (Klug A., 2010, Annu Rev Biochem 79, 213-231). Mutating certain amino acids in the zinc finger recognition surface altering the binding specificity of ZF modules resulted in defined ZF building blocks for most of 5′-GNN-3′, 5′-CNN-3′, 5′-ANN-3′, and some 5′-TNN-3′ codons (e.g. so-called Barbas modules, see Gonzalez B., 2010, Nat Protoc 5, 791-810). While early work on artificial transcription factors concentrated on a rational design based on combining preselected zinc fingers with a known 3 bp target sequence, the realization of a certain context specificity of zinc fingers necessitated the generation of large zinc finger libraries which are interrogated using sophisticated methods such as bacterial or yeast one hybrid, phage display, compartmentalized ribosome display or in vivo selection using FACS analysis.

Using such artificial zinc finger proteins, DNA loci within the human genome can be targeted with high specificity. Thus, these zinc finger proteins are ideal tools to transport protein domains with transcription-modulatory activity to specific promoter sequences resulting in the modulation of expression of a gene of interest. Suitable domains for the silencing of transcription are the Krueppel-associated domain (KRAB) as N-Terminal (SEQ ID NO: 1) or C-terminal (SEQ ID NO: 2) KRAB domain, the Sin3-interacting domain (SID, SEQ ID NO: 3) and the ERF repressor domain (ERD, SEQ ID NO: 4), while activation of gene transcription is achieved through Herpes Virus Simplex VP16 (SEQ ID NO: 5) or VP64 (tetrameric repeat of VP16, SEQ ID NO: 6) domains (Beerli R. R. et al., 1998, Proc Natl Acad Sci USA 95, 14628-14633). Additional domains considered to confer transcriptional activation are CJ7 (SEQ ID NO: 7), p65-TA1 (SEQ ID NO: 8), SAD (SEQ ID NO: 9), NF-1 (SEQ ID NO: 10), AP-2 (SEQ ID NO: 11), SP1-A (SEQ ID NO: 12), SP1-B (SEQ ID NO: 13), Oct-1 (SEQ ID NO: 14), Oct-2 (SEQ ID NO: 15), Oct-2_{—}5x (SEQ ID NO: 16), MTF-1 (SEQ ID NO: 17), BTEB-2 (SEQ ID NO: 18) and LKLF (SEQ ID NO: 19). In addition, transcriptionally active domains of proteins defined by gene ontology GO: 0001071 (http://amigo.geneontology.org/cgi-bin/amigo/term_details?term=GO:0001071) are considered to achieve transcriptional regulation of target proteins. Fusion proteins comprising engineered zinc finger proteins as well as regulatory domains are refered to as artificial transcription factors.

While small molecule drugs are not always able to selectively target a certain member of a given protein family due to the high conservation of specific features, biologicals based on naturally occurring or engineered proteins offer great specificity as shown for antibody-based novel drugs. However, virtually all biologicals to date act extracellularly. Especially above mentioned artificial transcription factors would be suitable to influence gene transcription in a therapeutically useful way. However, the delivery of such factors to the site of action—the nucleus—is not easily achieved, thus hampering the usefulness of therapeutic artificial transcription factor approaches, e.g. by relaying on retroviral delivery with all the drawbacks of this method such as immunogenicity and the potential for cellular transformation (Lund C. V. et al., 2005, Mol Cell Biol 25, 9082-9091).

So called protein transduction domains (PTDs) were shown to promote protein translocation across the plasma membrane into the cytosol/nucleoplasm. Short peptides such as the HIV derived TAT peptide (SEQ ID NO: 20) and others were shown to induce a cell-type independent macropinocytotic uptake of cargo proteins (Wadia J. S. et al., 2004, Nat Med 10, 310-315). Upon arrival in the cytosol, such fusion proteins were shown to have biological activity. Interestingly, even misfolded proteins can become functional following protein transduction most likely through the action of intracellular chaperones.

Nuclear receptors are a protein superfamily of ligand-activated transcription factors. They are, unlike most other cellular membrane-anchored receptors, soluble proteins localized to the cytosol or the nucleoplasm. Upon ligand binding and subsequent dimerization, nuclear receptors are capable of acting as transcription factors through DNA-binding and the modulation of gene expression. Ligands for nuclear receptors are lipophilic molecules, among them steroid and thyroid hormones, fatty and bile acids, retinoic acid, vitamin D3 and prostaglandins (McEwan I. J., Methods in Molecular Biology: The Nuclear Receptor Superfamily, 505, 3-17). Upon ligand binding, nuclear receptors dimerize, thus triggering binding to specific transcription-factor-specific DNA response elements inside ligand-responsive gene promoters causing either activation or repression of gene expression. Given that nuclear receptors are responsible for mediating the activity of many broad-acting hormones such as steroids and important metabolites, the miss- and dysfunction of nuclear receptors is involved in the natural history of many disorders.

Using agonists or antagonists to modulate the activity of nuclear receptors is employed for therapeutic purposes. Modulation of glucocorticoid receptor (GR) function using corticosteroids such as agonistic dexamethasone is common clinical practice for influencing inflammatory diseases. Another modulation of nuclear receptor activity is exemplified in oral contraception, wherein activation of the estrogen receptor (ESR1/ER) and the progesterone receptor is used to prevent egg fertilization in women. In another example, blocking the androgen receptor (AR) using anti-androgens such as flutamide or bicalutamide proved useful for the treatment of AR-dependent prostate cancers. Furthermore, blockage of the estrogen receptor by blocking estrogen synthesis and thus the availability of estrogen is a standard treatment for breast cancer in women or gynaecomastia in men.

### SUMMARY OF THE INVENTION

The invention relates to an artificial transcription factor comprising a polydactyl zinc finger protein targeting specifically a promoter region of a nuclear receptor gene fused to an inhibitory or activatory protein domain, a nuclear localization sequence, and a protein transduction domain, and to pharmaceutical compositions comprising such an artificial transcription factor. Furthermore the invention relates to the use of such artificial transcription factors for modulating the cellular response to nuclear receptor ligands, and in treating diseases modulated by the binding of specific effectors to such nuclear receptors.

In a particular embodiment, the promoter region of the nuclear receptor gene is the androgen receptor promoter (SEQ ID NO: 21). In this particular embodiment the invention relates to an artificial transcription factor targeting the androgen receptor promoter for use in influencing the cellular response to testosterone, for lowering or increasing androgen receptor levels, and for use in the treatment of diseases modulated by testosterone. Likewise the invention relates to a method of treating a disease modulated by testosterone comprising administering a therapeutically effective amount of an artificial transcription factor of the invention targeting the androgen receptor promoter to a patient in need thereof.

In another particular embodiment, the promoter region of the nuclear receptor gene is the estrogen receptor promoter (SEQ ID NO: 22). In this particular embodiment the invention relates to such an artificial transcription factor targeting the estrogen receptor promoter for use in influencing the cellular response to estrogen, for lowering or increasing estrogen receptor levels, and for use in the treatment of diseases modulated by estrogen. Likewise the invention relates to a method of treating a disease modulated by estrogen comprising administering a therapeutically effective amount of an artificial transcription factor of the invention targeting the estrogen receptor promoter to a patient in need thereof.

In yet another particular embodiment, the promoter region of the nuclear receptor gene is the glucocorticoid receptor promoter (SEQ ID NO: 23). In this particular embodiment the invention relates to an artificial transcription factor targeting the glucocorticoid receptor promoter for use in influencing the cellular response to glucocorticoids, for lowering or increasing glucocorticoid receptor levels, and for use in the treatment of diseases modulated by glucocorticoids, in particular for use in the treatment of eye diseases modulated by glucocorticoids. Likewise the invention relates to a method of treating a disease modulated by glucocorticoids comprising administering a therapeutically effective amount of an artificial transcription factor of the invention targeting the glucocorticoid receptor promoter to a patient in need thereof.

The invention further relates to nucleic acids coding for an artificial transcription factor of the invention, vectors comprising these, and host cells comprising such vectors.

### BRIEF DESCRIPTION OF THE FIGURES

FIG. 1: Modulating Gene Expression Using Transducible Artificial Transcription Factors

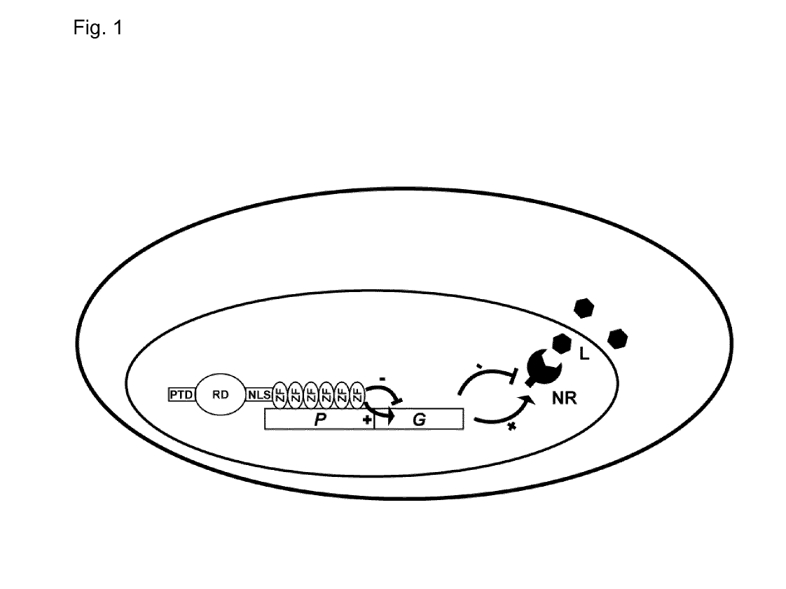

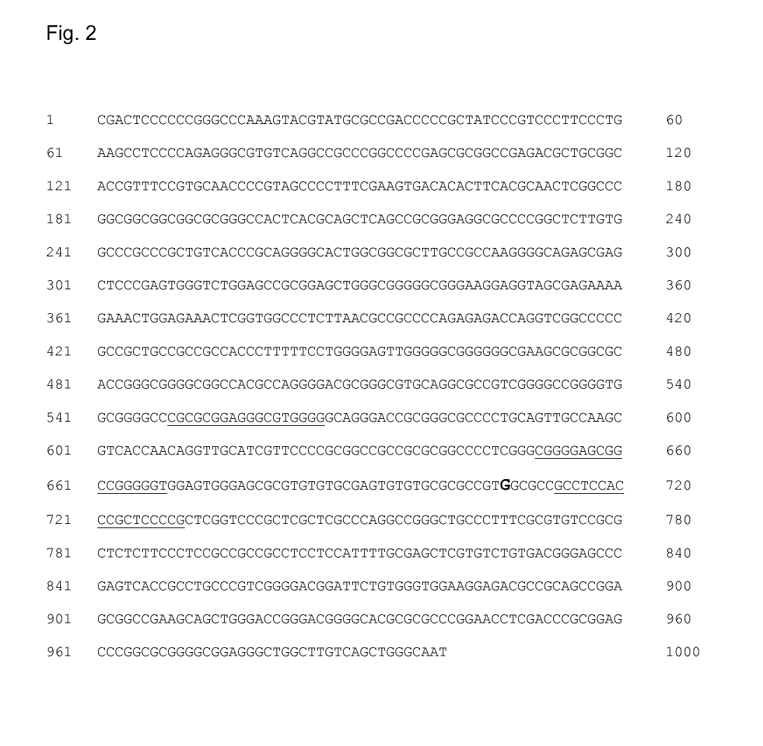

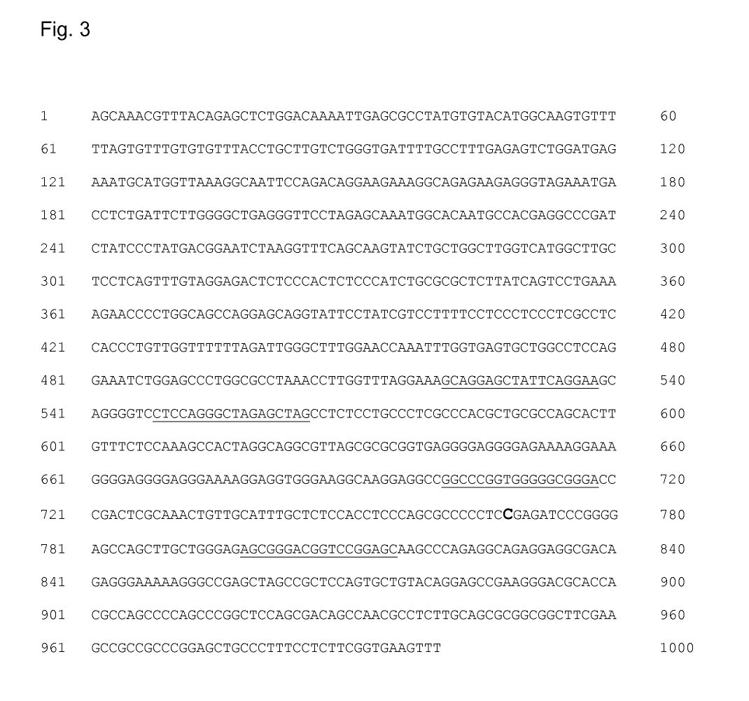

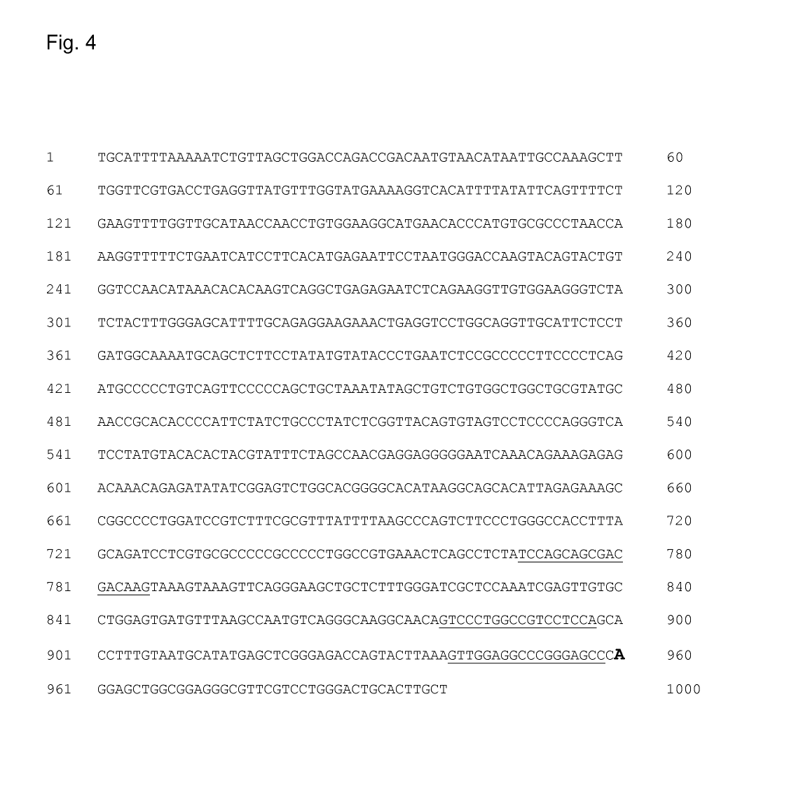

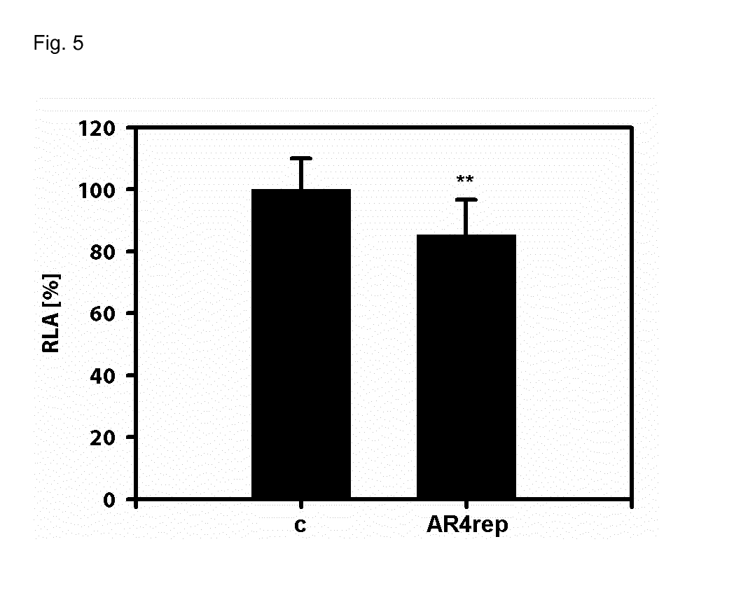

An artificial transcription factor containing a hexameric zinc finger (ZF) protein targeting specifically a promoter (P) region of a nuclear receptor gene (G) fused to an inhibitory/activatory domain (RD=regulatory domain) as well as a nuclear localization sequence (NLS) is transported into cells by the action of a protein transduction domain (PTD) such as TAT or others. Depending on the transcription-regulatory domain, receptor gene expression is either increased (+) or suppressed (−) resulting in an enhanced or diminished expression of a nuclear receptor (NR) and therefore enhanced or diminished cellular response to nuclear receptor ligand (L).

FIG. 2: Human Glucocorticoid Receptor Promoter and Artificial Transcription Factor Target Sites

Shown is the 5′ untranslated region of the glucocorticoid receptor promoter (SEQ ID NO: 21). Highlighted are the transcription start site (marked bold, position 707) and three binding sites for artificial transcription factors of the invention (underlined).

FIG. 3: Human Androgen Receptor Promoter and Artificial Transcription Factor Target Sites

Shown is the 5′ untranslated region of the androgen receptor promoter (SEQ ID NO: 22). Highlighted are the transcription start site (marked bold, position 768) and four binding sites for artificial transcription factors of the invention (underlined).

FIG. 4: Human Estrogen Receptor Promoter and Artificial Transcription Factor Target Sites

Shown is the 5′ untranslated region of the estrogen receptor promoter (SEQ ID NO: 23). Highlighted are the transcription start site (marked bold, position 960) and three binding sites for artificial transcription factors of the invention (underlined).

FIG. 5: AR4 Rep is Able to Suppress Gene Expression in a Luciferase Reporter Assay

HEK 293 Flpin TRex cells containing a reporter construct consisting of Gaussia luciferase under control of a hybrid CMV/AR_TS4 promoter as well as secreted alkaline phosphatase under control of the constitutive CMV promoter were treated with 1 μM AR4rep for 2 hours in OptiMEM media. Treatment with an unrelated artificial transcription factor ATFControl (SEQ ID NO: 24) served as control (labeled c). Luciferase as well as secreted alkaline phosphatase activity was measured 24 hours after AR4rep treatment. Luciferase activity normalized to secreted alkaline phosphatase activity was expressed as relative luciferase activity (RLA) in percent of control. Statistical significance was analyzed using two-tailed, unpaired Student's t-test. P-Value<0.01 is marked with **.

### DETAILED DESCRIPTION OF THE INVENTION

The invention relates to an artificial transcription factor comprising a polydactyl zinc finger protein targeting specifically a promoter region of a nuclear receptor gene fused to an inhibitory or activatory protein domain, a nuclear localization sequence, and a protein transduction domain, and to pharmaceutical compositions comprising such an artificial transcription factor.

In contrast to almost all other cellular receptors that are membrane-anchored and consist or contain membrane-spanning proteins, nuclear receptors are soluble proteins incorporating ligand binding and transcription factor activity in one polypeptide. Nuclear receptors are either localized to the cytosol or the nucleoplasm, where they are activated upon ligand binding, dimerize and become active transcription factors regulating a vast array of transcriptional programs. Unlike above mentioned membrane-anchored receptors that bind their ligands outside the cell and transduce the signal across the plasma membrane into the cell, nuclear receptors bind lipophilic ligands that are capable of crossing the plasma membrane to gain access to their cognate receptor. In addition, most membrane-bound receptors rely on intricate signal amplification mechanisms before the intended cellular outcome is achieved. Nuclear receptors, on the other hand, directly convert the binding of a ligand into a cellular response.

Treatment of many diseases is based on modulating nuclear receptor signaling. Examples are inflammatory processes, wherein glucocorticoids activate the glucocorticosteriod receptor, prostate cancer, wherein antagonists of androgen receptor possess beneficial therapeutic effect, or breast cancer, wherein blocking estrogen receptor signaling proves useful. Traditionally, small molecules either in the form of nuclear receptor agonist or antagonists are used to impact receptor signaling for therapeutic purposes. However, nuclear receptor signaling can also be influenced by direct modulation of nuclear receptor protein expression, and such modulation is the subject of the present invention.

Nuclear receptors considered in the present invention are human nuclear receptors encoded by the human genes AR, ESR1, ESR2, ESRRA, ESRRB, ESRRG, HNF4A, HNF4G, NR0B1, NR0B2, NR1D1, NR1D2, NR1H2, NR1H3, NR1H4, NR1I2, NR1I3, NR2C1, NR2C2, NR2E1, NR2E3, NR2F1, NR2F2, NR2F6, NR3C1, NR3C2, NR4A1, NR4A2, NR4A3, NR5A1, NR5A2, NR6A1, PGR, PPARA, PPARD, PPARG, RARA, RARB, RARG, RORA, RORB, RORC, RXRA, RXRB, RXRG, THRA, THRB and VDR.

Further considered are non-human nuclear receptors, for example porcine, equine, bovine, feline, canine, or murine transcription factors, encoded by genes related to the mentioned human nuclear receptor genes.

According to the state of the art, intracellular expression of artificial transcription factors is accomplished using viral transduction. Viral vectors have exceptionally high potential for immunogenicity, thus limiting their use in repeated application of a certain treatment. Due to the high conservation of zinc finger modules such an immune reaction will be minor or absent following application of artificial transcription factors of the invention, or might be avoided or further minimized by small changes to the overall structure eliminating immunogenicity while still retaining target site binding and thus function. Furthermore, modification of artificial transcription factors of the invention with polyethylene glycol is considered to reduce immunogenicity. In addition, application of artificial transcription factors of the invention to immune privileged organs such as the eye and the brain will avoid any immune reaction, and induce whole body tolerance to the artificial transcription factors. For the treatment of chronic diseases outside of immune privileged organs, induction of immune tolerance through prior intraocular injection is considered.

Classes of small molecules traditionally used as pool for therapeutic agents are not suitable for targeted modulation of gene expression. Thus, many promising drug targets and associated diseases are not amenable to classical pharmaceutical approaches. This is especially true for transcription factors that are considered as not drugable. In contrast, artificial transcription factors of the invention all belong to the same substance class with a highly defined overall composition. Two hexameric zinc finger protein-based artificial transcription factors targeting two very diverse promoter sequences still have a minimal amino acid sequence identity of 85% with an overall similar tertiary structure and can be generated via a standardized method (as described below) in a fast and economical manner. Thus, artificial transcription factors of the invention combine, in one class of molecules, exceptionally high specificity for a very wide and diverse set of targets with overall similar composition. In addition, formulation of artificial transcription factors of the invention into drugs can rely on previous experience further expediting the drug development process.

Protein transduction domain (PTD) mediated, intracellular delivery of artificial transcription factors is a new way of taking advantage of the high selectivity of biologicals to target receptor molecules in a novel fashion. While conventional drugs modulate the activity of certain receptors, artificial transcription factors alter the availability of these proteins. And since artificial transcription factors are tailored to act specifically on the promoter region of such receptor genes, the invention allows selectively targeting even closely related proteins. This is based on the only loose conservation of the promoter regions even of closely related proteins. The protein transduction domain-mediated delivery of artificial transcription factors is useful to modulate the cellular response to ligands of nuclear receptors.

Protein transduction domains considered are HIV TAT, the peptide mT02 (SEQ ID NO: 25), the peptide mT03 (SEQ ID NO: 26), the R9 peptide (SEQ ID NO: 27), the ANTP domain (SEQ ID NO: 28) or other peptides capable of transporting cargo across the plasma membrane.

The invention also relates the use of such artificial transcription factors in treating diseases modulated by the binding of nuclear receptor ligands to nuclear receptors, for which the polydactyl zinc finger protein is specifically targeting the promoter region of a nuclear receptor gene. Likewise the invention relates to a method of treating diseases comprising administering a therapeutically effective amount of an artificial transcription factor to a patient in need thereof, wherein the disease to be treated is modulated by the binding of specific effectors to nuclear receptors, for which the polydactyl zinc finger protein is specifically targeting the receptor gene promoter.

Polydactyl zinc finger proteins considered are tetrameric, pentameric, hexameric, heptameric or octameric zinc finger proteins. “Tetrameric”, “pentameric”, “hexameric”, “heptameric” and “octameric” means that the zinc finger protein consists of four, five, six, seven or eight partial protein structures, respectively, each of which has binding specificity for a particular nucleotide triplet. Preferably the artificial transcription factors comprise hexameric zinc finger proteins.

Selection of Target Sites within a Given Promoter Region

Target site selection is crucial for the successful generation of a functional artificial transcription factor. For an artificial transcription factor to modulate nuclear receptor gene expression in vivo, it must bind its target site in the genomic context of the nuclear receptor gene. This necessitates the accessibility of the DNA target site, meaning chromosomal DNA in this region is not tightly packed around histones into nucleosomes and no DNA modifications such as methylation interfere with artificial transcription factor binding. While large parts of the human genome are tightly packed and transcriptionally inactive, the immediate vicinity of the transcriptional start site (−1000 to +200 bp) of an actively transcribed gene must be accessible for endogenous transcription factors and the transcription machinery such as RNA polymerases. Thus, selecting a target site in this area of any given target gene will greatly enhance the success rate for the generation of an artificial transcription factor with the desired function in vivo.

Selection of Target Sites within the Human Glucocorticoid, Androgen and Estrogen Receptor Gene Promoters

The promoter region comprising 1000 bp including the transcriptional start site of the human glucocorticoid, androgen and estrogen receptor open reading frame (FIGS. 2, 3 and 4) was analyzed for the presence of potential 18 bp target sites with the general composition of (G/C/ANN)₆, wherein G is the nucleotide guanine, C the nucleotide cytosine, A the nucleotide adenine and N stands for each of the four nucleotide guanine, cytosine, adenine and thymine. Three to four target sites in each promoter were selected based on their position relative to the transcription start site. The target sites found in the glucocorticoid receptor gene promoter are GR_TS1 (SEQ ID NO: 29), GR_TS2 (SEQ ID NO: 30), GR_TS3 (SEQ ID NO: 31), and the target sites for the androgen receptor are AR_TS1 (SEQ ID NO: 32), AR_TS2 (SEQ ID NO: 33), AR_TS3 (SEQ ID NO: 34) and AR_TS4 (SEQ ID NO: 35). The target sites identified in the estrogen receptor gene promoter are ER_TS1 (SEQ ID NO: 36), ER_TS2 (SEQ ID NO: 37) and ER_TS3 (SEQ ID NO: 38). Considered are also target sites of the general composition (G/C/ANN)₅ and (G/C/ANN)₆ chosen from the regulatory region of the glucocorticoid receptor, the estrogen receptor and the androgen receptor 2000 bp upstream of the transcription start.

Transducible Artificial Transcription Factors Targeting the Glucocorticoid Receptor Promoter

Specific hexameric zinc finger proteins were composed of the so called Barbas zinc finger module set (Gonzalez B., 2010, Nat Protoc 5, 791-810) using the ZiFit software v3.3 (Sander J. D., Nucleic Acids Research 35, 599-605). To generate activating transducible artificial transcription factors targeting the glucocorticoid receptor, hexameric zinc finger proteins ZFP-GR1 (SEQ ID NO: 39) targeting GR_TS1, ZFP-GR2 (SEQ ID NO: 40) targeting GR_TS2, and ZFP-GR3 (SEQ ID NO: 41) targeting GR_TS3 were fused to the protein transduction domain TAT as well as the transcription activating domain VP64 yielding artificial transcription factors GR1akt (SEQ ID NO: 42), GR2akt (SEQ ID NO: 43) and GR3akt (SEQ ID NO: 44). To generate transducible artificial transcription factors with negative regulatory activity, hexameric zinc finger proteins ZFP-GR1 to ZFP-GR3 were fused to the protein transduction domain TAT as well as the transcription repressing domain KRAB yielding artificial transcription factors GR1rep (SEQ ID NO: 45), GR2rep (SEQ ID NO: 46) and GR3rep (SEQ ID NO: 47).

Transducible Artificial Transcription Factors Targeting the Androgen Receptor Promoter

Specific hexameric zinc finger proteins were composed from the so called Barbas zinc finger module set using the ZiFit software v3.3. Additional zinc finger proteins targeting the AR promoter were selected using yeast one hybrid screening. To generate activating transducible artificial transcription factors targeting the androgen receptor, hexameric zinc finger proteins ZFP-AR1 (SEQ ID NO: 48) targeting AR_TS1, ZFP-AR2 (SEQ ID NO: 49) targeting AR_TS2, ZFP-AR3 (SEQ ID NO: 50) targeting AR_TS3, and ZFP-AR4 (SEQ ID NO: 51), ZFP-AR5 (SEQ ID NO: 52) and ZFP-AR6 (SEQ ID NO: 53) targeting AR_TS4 were fused to the protein transduction domain TAT as well as the transcription activating domain VP64 yielding artificial transcription factors AR1akt (SEQ ID NO: 54), AR2akt (SEQ ID NO: 55), AR3akt (SEQ ID NO: 56), AR4akt (SEQ ID NO: 57), AR5akt (SEQ ID NO: 58) and AR6akt (SEQ ID NO: 59). To generate transducible artificial transcription factor with negative-regulatory activity, hexameric zinc finger proteins ZFP-AR1 to ZFP-AR6 were fused to the protein transduction domain TAT as well as the transcription repressing domain SID yielding artificial transcription factors AR1 rep (SEQ ID NO: 60), AR2rep (SEQ ID NO: 61), AR3rep (SEQ ID NO: 62), AR4rep (SEQ ID NO: 63), AR5rep (SEQ ID NO: 64) and AR6rep (SEQ ID NO: 65).

Transducible Artificial Transcription Factors Targeting the Estrogen Receptor Promoter

Specific hexameric zinc finger proteins were composed of the so called Barbas zinc finger module set using the ZiFit software v3.3. To generate activating transducible artificial transcription factors targeting the estrogen receptor, hexameric zinc finger proteins ZFP-ER1 (SEQ ID NO: 66) targeting ER_TS1, ZFP-ER2 (SEQ ID NO: 67) targeting ER_TS2, and ZFP-ER3 (SEQ ID NO: 68) targeting ER_TS3 were fused to the protein transduction domain TAT as well as the transcription activating domain VP64 yielding artificial transcription factors ER1akt (SEQ ID NO: 69), ER2akt (SEQ ID NO: 70) and ER3akt (SEQ ID NO: 71). To generate transducible artificial transcription factors with negative-regulatory activity, hexameric zinc finger proteins ZFP-ER1 to ZFP-ER3 were fused to the protein transduction domain TAT as well as the transcription repressing domain SID yielding artificial transcription factors ER1rep (SEQ ID NO: 72), ER2rep (SEQ ID NO: 73) and ER3rep (SEQ ID NO: 74).

The artificial transcription factors targeting glucocorticoid, androgen or estrogen receptor according to the invention also comprise a zinc finger protein based on the zinc finger module composition as disclosed in SEQ ID NO 39 to 41, 48 to 53 and 66 to 68, respectively, wherein up to four individual zinc finger modules are exchanged against other zinc finger modules with alternative binding characteristic to modulate the binding of the artificial transcription factor to its target sequence.

Considered are also artificial transcription factors of the invention containing pentameric, hexameric, heptameric or octameric zinc finger proteins, wherein individual zinc finger modules are exchanged to improve binding affinity towards target sites of the respective nuclear receptor promoter gene or to alter the immunological profile of the zinc finger protein for improved tolerability.

The artificial transcription factors targeting the nuclear receptors glucocorticoid, androgen and or estrogen receptor according to the invention also comprise a zinc finger protein based on the zinc finger module composition as disclosed in SEQ ID NO 39 to 41, 48 to 53 and 66 to 68, respectively, wherein individual amino acids are exchanged in order to minimize potential immunogenicity while retaining binding affinity to the intended target site.

The artificial transcription factor of the present invention might also contain other transcriptionally active protein domains of proteins defined by gene ontology GO:0001071 such as N-terminal KRAB, C-terminal KRAB, SID and ERD domains, preferably SID. Activatory protein domains considered are the transcriptionally active domains of proteins defined by gene ontology GO:0001071, such as VP16 or VP64 (tetrameric repeat of VP16), preferably VP64.

Further, the artificial transcription factors of the invention comprise a nuclear localization sequence (NLS). Nuclear localization sequences considered are amino acid motifs conferring nuclear import through binding to proteins defined by gene ontology GO:0008139, for example clusters of basic amino acids containing a lysine residue (K) followed by a lysine (K) or arginine residue (R), followed by any amino acid (X), followed by a lysine or arginine residue (K-K/R-X-K/R consensus sequence, Chelsky D. et al., 1989 Mol Cell Biol 9, 2487-2492) or the SV40 NLS (SEQ ID NO: 75), with the SV40 NLS being preferred.

Artificial transcription factors directed to a promoter region of a nuclear receptor gene, but without the protein transduction domain, are also a subject of the invention. They are intermediates for the artificial transcription factors of the invention as defined hereinbefore. Particular embodiments of such artificial transcription factors directed to a promoter region of a nuclear receptor gene, but without the protein transduction domain, are artificial transcription factors directed to the androgen receptor gene promoter, and artificial transcription factors directed to the estrogen receptor gene promoter, all without the protein transduction domain.

Further considered are alternative delivery methods for artificial transcription factors of the invention in form of nucleic acids transferred by transfection or via viral vectors such as, but not limited to, herpes virus-, adeno virus- and adeno-associated virus-based vectors.

The domains of the artificial transcription factors of the invention may be connected by short flexible linkers. A short flexible linker has 2 to 8 amino acids, preferably glycine and serine. A particular linker considered is GGSGGS (SEQ ID NO: 76). Artificial transcription factors may further contain markers to ease their detection and processing.

Assessment of Glucocorticoid Receptor Modulation Following Artificial Transcription Factor Treatment

HeLa cells treated with a glucocorticoid receptor promoter specific negative regulatory artificial transcription factor are compared to control treated cells in terms of transcriptional induction following dexamethasone treatment. Using quantitative RT-PCR, the expression levels of glucocorticoid receptor target genes TSC22D3, IGFBP1 and IRF8 are measured. A decreased expression of these glucocorticoid responsive genes in artificial transcription factor treated cells compared to control cells is proof for the regulatory activity of the glucocorticoid receptor-specific artificial transcription factor.

Assessment of Androgen Receptor Modulation Following Artificial Transcription Factor Treatment

Cells expressing the androgen receptor treated with an androgen receptor promoter specific negative regulatory artificial transcription factor are compared to control treated cells in terms of transcriptional induction following testosterone treatment. Using quantitative RT-PCR, the expression levels of androgen receptor target genes PSA, SPAK and TMPRSS2 are measured. A decreased expression of these androgen responsive genes in artificial transcription factor treated cells compared to control cells is proof for the regulatory activity of the androgen receptor-specific artificial transcription factor.

Assessment of Estrogen Receptor Modulation Following Artificial Transcription Factor Treatment

Cells expressing the estrogen receptor treated with an estrogen receptor promoter specific negative regulatory artificial transcription factor are compared to control treated cells in terms of transcriptional induction following estradiol treatment. Using quantitative RT-PCR, the expression levels of estrogen receptor target genes bcl-2, ovalbumin, c-fos, collagenase and oxytocin are measured. A decreased expression of these estradiol responsive genes in artificial transcription factor treated cells compared to control cells is proof for the regulatory activity of the estrogen receptor-specific artificial transcription factor.

Assessment of AR4rep Activity in a Luciferase Reporter Assay

A reporter cell line based on HEK 293 Flpin TRex cells containing Gaussia luciferase under control of a hybrid CMV/AR_TS4 promoter and secreted alkaline phosphatase under control of the constitutive CMV promoter was used to assess activity of AR4rep. As shown in FIG. 5, treatment of such cells with AR4rep caused a decrease in luciferase activity compared to control treated cells.

Attachment of a Polyethylene Glycol Residue

The covalent attachment of a polyethylene glycol residue (PEGylation) to an artificial transcription factor of the invention is considered to increase solubility of the artificial transcription factor, to decrease its renal clearance, and control its immunogenicity. Considered are amine as well as thiol reactive polyethylene glycols ranging in size from 1 to 40 Kilodalton. Using thiol reactive polyethylene glycols, site-specific PEGylation of the artificial transcription factors is achieved. The only essential thiol group containing amino acids in the artificial transcription factors of the invention are the cysteine residues located in the zinc finger modules essential for zinc coordination. These thiol groups are not accessible for PEGylation due their zinc coordination, thus, inclusion of one or several cysteine residues into the artificial transcription factors of the invention provides free thiol groups for PEGylation using thiol-specific polyethylene glycol reagents.

Pharmaceutical Compositions

The present invention relates also to pharmaceutical compositions comprising an artificial transcription factor as defined above. Pharmaceutical compositions considered are compositions for parenteral systemic administration, in particular intravenous administration, compositions for inhalation, and compositions for local administration, in particular ophthalmic-topical administration, e.g. as eye drops, or intravitreal, subconjunctival, parabulbar or retrobulbar administration, to warm-blooded animals, especially humans. Particularly preferred are eye drops and compositions for intravitreal, subconjunctival, parabulbar or retrobulbar administration. The compositions comprise the active ingredient alone or, preferably, together with a pharmaceutically acceptable carrier. Further considered are slow-release formulations. The dosage of the active ingredient depends upon the disease to be treated and upon the species, its age, weight, and individual condition, the individual pharmacokinetic data, and the mode of administration.

Further considered are pharmaceutical compositions useful for oral delivery, in particular compositions comprising suitably encapsulated active ingredient, or otherwise protected against degradation in the gut. For example, such pharmaceutical compositions may contain a membrane permeability enhancing agent, a protease enzyme inhibitor, and be enveloped by an enteric coating.

The pharmaceutical compositions comprise from approximately 1% to approximately 95% active ingredient. Unit dose forms are, for example, ampoules, vials, inhalers, eye drops and the like.

The pharmaceutical compositions of the present invention are prepared in a manner known per se, for example by means of conventional mixing, dissolving or lyophilizing processes.

Preference is given to the use of solutions of the active ingredient, and also suspensions or dispersions, especially isotonic aqueous solutions, dispersions or suspensions which, for example in the case of lyophilized compositions comprising the active ingredient alone or together with a carrier, for example mannitol, can be made up before use. The pharmaceutical compositions may be sterilized and/or may comprise excipients, for example preservatives, stabilizers, wetting agents and/or emulsifiers, solubilizers, salts for regulating osmotic pressure and/or buffers and are prepared in a manner known per se, for example by means of conventional dissolving and lyophilizing processes. The said solutions or suspensions may comprise viscosity-increasing agents, typically sodium carboxymethylcellulose, carboxymethylcellulose, dextran, polyvinylpyrrolidone, or gelatins, or also solubilizers, e.g. Tween 80™ (polyoxyethylene(20)sorbitan mono-oleate).

Suspensions in oil comprise as the oil component the vegetable, synthetic, or semi-synthetic oils customary for injection purposes. In respect of such, special mention may be made of liquid fatty acid esters that contain as the acid component a long-chained fatty acid having from 8 to 22, especially from 12 to 22, carbon atoms. The alcohol component of these fatty acid esters has a maximum of 6 carbon atoms and is a monovalent or polyvalent, for example a mono-, di- or trivalent, alcohol, especially glycol and glycerol. As mixtures of fatty acid esters, vegetable oils such as cottonseed oil, almond oil, olive oil, castor oil, sesame oil, soybean oil and groundnut oil are especially useful.

The manufacture of injectable preparations is usually carried out under sterile conditions, as is the filling, for example, into ampoules or vials, and the sealing of the containers.

For parenteral administration, aqueous solutions of the active ingredient in water-soluble form, for example of a water-soluble salt, or aqueous injection suspensions that contain viscosity-increasing substances, for example sodium carboxymethylcellulose, sorbitol and/or dextran, and, if desired, stabilizers, are especially suitable. The active ingredient, optionally together with excipients, can also be in the form of a lyophilizate and can be made into a solution before parenteral administration by the addition of suitable solvents.

Compositions for inhalation can be administered in aerosol form, as sprays, mist or in form of drops. Aerosols are prepared from solutions or suspensions that can be delivered with a metered-dose inhaler or nebulizer, i.e. a device that delivers a specific amount of medication to the airways or lungs using a suitable propellant, e.g. dichlorodifluoro-methane, trichlorofluoromethane, dichlorotetrafluoroethane, carbon dioxide or other suitable gas, in the form of a short burst of aerosolized medicine that is inhaled by the patient. It is also possible to provide powder sprays for inhalation with a suitable powder base such as lactose or starch.

Eye drops are preferably isotonic aqueous solutions of the active ingredient comprising suitable agents to render the composition isotonic with lacrimal fluid (295-305 mOsm/l). Agents considered are sodium chloride, citric acid, glycerol, sorbitol, mannitol, ethylene glycol, propylene glycol, dextrose, and the like. Furthermore the composition comprise buffering agents, for example phosphate buffer, phosphate-citrate buffer, or Tris buffer (tris(hydroxymethyl)-aminomethane) in order to maintain the pH between 5 and 8, preferably 7.0 to 7.4. The compositions may further contain antimicrobial preservatives, for example parabens, quaternary ammonium salts, such as benzalkonium chloride, polyhexamethylene biguanidine (PHMB) and the like. The eye drops may further contain xanthan gum to produce gel-like eye drops, and/or other viscosity enhancing agents, such as hyaluronic acid, methylcellulose, polyvinylalcohol, or polyvinylpyrrolidone.

Use of Artificial Transcription Factors in a Method of Treatment

Furthermore, the invention relates an artificial transcription factor assembled as to target the promoter region of a nuclear receptor as described above for use in influencing the cellular response to the nuclear receptor ligand, for lowering or increasing the levels of the nuclear receptor, and for use in the treatment of diseases modulated by such nuclear receptors. Likewise, the invention relates to a method of treating diseases modulated by a nuclear receptor ligand comprising administering a therapeutically effective amount of an artificial transcription factor directed to a nuclear receptor promoter to a patient in need thereof.

Diseases modulated by ligands of nuclear receptors are, for example, adrenal insufficiency, adrenocortical insufficiency, alcoholism, Alzheimer's disease, androgen insensitivity syndrome, anorexia nervosa, aortic aneurysm, aortic valve sclerosis, arthritis, asthma, atherosclerosis, attention deficit hyperactivity disorder, autism, azoospermia, biliary primary cirrhosis, bipolar disorder, bladder cancer, bone cancer, breast cancer, cardiovascular disease, cardiovascular myocardial infarction, celiac disease, cholestasis, chronic kidney failure and metabolic syndrome, cirrhosis, cleft palate, colorectal cancer, congenital adrenal hypoplasia, coronary heart disease, cryptorchidism, deep vein thrombosis, dementia, depression, diabetic retinopathy, dry eye disease, endometriosis, endometrial cancer, enhanced S-cone syndrome, essential hypertension, familial partial lipodystrophy, glioblastoma, glucocorticoid resistance, Graves' Disease, high serum lipid levels, hyperapobetalipoproteinemia, hyperlipidemia, hypertension, hypertriglyceridemia, hypogonadotropic hypogonadism, hypospadias, infertility, inflammatory bowel disease, insulin resistance, ischemic heart disease, liver steatosis, lung cancer, lupus erythematosus, major depressive disorder, male breast cancer, metabolic plasma lipid levels, metabolic syndrome, migraine, mulitple sclerosis, myocardial infarct, nephrotic syndrome, non-Hodgkin's lymphoma, obesity, osteoarthritis, osteopenia, osteoporosis, ovarian cancer, Parkinson's disease, preeclampsia, progesterone resistance, prostate cancer, pseudohypoaldosteronism, psoriasis, psychiatric schizophrenia, psychosis, retinitis pigmentosa-37, schizophrenia, sclerosing cholangitis, sex reversal, skin cancer, spinal and bulbar atrophy of Kennedy, susceptibility to myocardial infarction, susceptibility to psoriasis, testicular cancer, type I diabetes, type II diabetes, uterine cancer and vertigo.

Likewise, the invention relates to a method of treating a disease modulated by ligands of nuclear receptors comprising administering a therapeutically effective amount of an artificial transcription factor of the invention to a patient in need thereof. In particular, the invention relates to a method of treating adrenal insufficiency, adrenocortical insufficiency, alcoholism, Alzheimer's disease, androgen insensitivity syndrome, anorexia nervosa, aortic aneurysm, aortic valve sclerosis, arthritis, asthma, atherosclerosis, attention deficit hyperactivity disorder, autism, azoospermia, biliary primary cirrhosis, bipolar disorder, bladder cancer, bone cancer, breast cancer, cardiovascular disease, cardiovascular myocardial infarction, celiac disease, cholestasis, chronic kidney failure and metabolic syndrome, cirrhosis, cleft palate, colorectal cancer, congenital adrenal hypoplasia, coronary heart disease, cryptorchidism, deep vein thrombosis, dementia, depression, diabetic retinopathy, dry eye disease, endometriosis, endometrial cancer, enhanced S-cone syndrome, essential hypertension, familial partial lipodystrophy, glioblastoma, glucocorticoid resistance, Graves' Disease, high serum lipid levels, hyperapobeta-lipoproteinemia, hyperlipidemia, hypertension, hypertriglyceridemia, hypogonadotropic hypogonadism, hypospadias, infertility, inflammatory bowel disease, insulin resistance, ischemic heart disease, liver steatosis, lung cancer, lupus erythematosus, major depressive disorder, male breast cancer, metabolic plasma lipid levels, metabolic syndrome, migraine, multiple sclerosis, myocardial infarct, nephrotic syndrome, non-Hodgkin's lymphoma, obesity, osteoarthritis, osteopenia, osteoporosis, ovarian cancer, Parkinson's disease, preeclampsia, progesterone resistance, prostate cancer, pseudohypoaldosteronism, psoriasis, psychiatric schizophrenia, psychosis, retinitis pigmentosa-37, schizophrenia, sclerosing cholangitis, sex reversal, skin cancer, spinal and bulbar atrophy of Kennedy, susceptibility to myocardial infarction, susceptibility to psoriasis, testicular cancer, type I diabetes, type II diabetes, uterine cancer and vertigo, comprising administering an effective amount of an artificial transcription factor of the invention to a patient in need thereof. The effective amount of an artificial transcription factor of the invention depends upon the particular type of disease to be treated and upon the species, its age, weight, and individual condition, the individual pharmacokinetic data, and the mode of administration. For administration into the eye, a monthly vitreous injection of 0.5 to 1 mg is preferred. For systemic application, a monthly injection of 10 mg/kg is preferred. In addition, implantation of slow release deposits into the vitreous of the eye is also preferred.

Furthermore, the invention relates an artificial transcription factor directed to the androgen receptor as described above for use in influencing the cellular response to ligands of the androgen receptor, for lowering or increasing androgen receptor levels, and for the use in the treatment of diseases modulated by ligands of the androgen receptor.

Likewise the invention relates to a method of treating a disease modulated by ligands of the androgen receptor comprising administering a therapeutically effective amount of an artificial transcription factor of the invention to a patient in need thereof. Diseases considered are prostate cancer, male breast cancer, ovarian cancer, colorectal cancer, endometrial cancer, testicular cancer, coronary artery disease, type I diabetes, diabetic retinopathy, obesity, androgen insensitivity syndrome, osteoporosis, osteoarthritis, type II diabetes, Alzheimer's disease, migraine, attention deficit hyperactivity disorder, depression, schizophrenia, azoospermia, endometriosis, and spinal and bulbar atrophy of Kennedy. In particular, upregulating AR levels is beneficial for the treatment of dry eye disease, while downregulation of AR levels is beneficial for the treatment of AR-blockage insensitive prostate cancers. The effective amount of an artificial transcription factor of the invention depends upon the particular type of disease to be treated and upon the species, its age, weight, and individual condition, the individual pharmacokinetic data, and the mode of administration. For administration into the eye, a monthly vitreous injection of 0.5 to 1 mg is preferred. For systemic application, a monthly injection of 10 mg/kg is preferred. In addition, implantation of slow release deposits into the vitreous of the eye is also preferred.

Furthermore, the invention relates an artificial transcription factor directed to the estrogen receptor as described above for use in influencing the cellular response to ligands of the estrogen receptor, for lowering or increasing estrogen receptor levels, and for the use in the treatment of diseases modulated by ligands of the estrogen receptor.

Likewise the invention relates to a method of treating a disease modulated by ligands of the estrogen receptor comprising administering a therapeutically effective amount of an artificial transcription factor of the invention to a patient in need thereof. Diseases considered are bone cancer, breast cancer, colorectal cancer, endometrial cancer, prostate cancer uterine cancer, alcoholism, migraine, aortic aneurysm, susceptibility to myocardial infarction, aortic valve sclerosis, cardiovascular disease, coronary artery disease, hypertension, deep vein thrombosis, Graves' Disease, arthritis, mulitple sclerosis, cirrhosis, hepatitis B, chronic liver disease, cholestasis, hypospadias, obesity, osteoarthritis, osteopenia, osteoporosis, Alzheimer's disease, Parkinson's disease, migraine, vertigo), anorexia nervosa, attention deficit hyperactivity disorder, dementia, depression, psychosis, endometriosis and infertility. In particular, downregulation of ER levels is beneficial for the treatment of hormone-dependent breast cancer. The effective amount of an artificial transcription factor of the invention depends upon the particular type of disease to be treated and upon the species, its age, weight, and individual condition, the individual pharmacokinetic data, and the mode of administration. For administration into the eye, a monthly vitreous injection of 0.5 to 1 mg is preferred. For systemic application, a monthly injection of 10 mg/kg is preferred. In addition, implantation of slow release deposits into the vitreous of the eye is also preferred.

Use of Artificial Transcription Factors in Animals

Furthermore the invention relates to the use of artificial transcription factors targeting nuclear receptors found in animals for the treatment of diseases modulated by dysfunction of such nuclear receptors. Preferably, the artificial transcription factors are directly applied in suitable compositions for topical applications to animals in need thereof.

### Examples

### Cloning of DNA Plasmids

For all cloning steps, restriction endonucleases and T4 DNA ligase are purchased from New England Biolabs. Shrimp Alkaline Phosphatase (SAP) is from Promega. The high-fidelity Platinum Pfx DNA polymerase (Invitrogen) is applied in all standard PCR reactions. DNA fragments and plasmids are isolated according to the manufacturer's instructions using NucleoSpin Gel and PCR Clean-up kit, NucleoSpin Plasmid kit, or NucleoBond Xtra Midi Plus kit (Macherey-Nagel). Oligonucleotides are purchased from Sigma-Aldrich. All relevant DNA sequences of newly generated plasmids were verified by sequencing (Microsynth).

Cloning of Hexameric Zinc Finger Protein Libraries for Yeast One Hybrid

Hexameric zinc finger protein libraries containing GNN and/or CNN and/or ANN binding zinc finger (ZF) modules are cloned according to Gonzalez B. et al. 2010, Nat Protoc 5, 791-810 with the following improvements. DNA sequences coding for GNN, CNN and ANN ZF modules were synthesized and inserted into pUC57 (GenScript) resulting in pAN1049 (SEQ ID NO: 77), pAN1073 (SEQ ID NO: 78) and pAN1670 (SEQ ID NO: 79), respectively. Stepwise assembly of zinc finger protein (ZFP) libraries is done in pBluescript SK (+) vector. To avoid insertion of multiple ZF modules during each individual cloning step leading to non-functional proteins, pBluescript (and its derived products containing 1ZFP, 2ZFPs, or 3ZFPs) and pAN1049, pAN1073 or pAN1670 are first incubated with one restriction enzyme and afterwards treated with SAP. Enzymes are removed using NucleoSpin Gel and PCR Clean-up kit before the second restriction endonuclease is added.

Cloning of pBluescript-1ZFPL is done by treating 5 μg pBluescript with Xhol, SAP and subsequently Spel. Inserts are generated by incubating 10 μg pAN1049 (release of 16 different GNN ZF modules) or pAN1073 (release of 15 different CNN ZF modules) or pAN1670 (release of 15 different ANN ZF modules) with Spel, SAP and subsequently Xhol. For generation of pBluescript-2ZFPL and pBluescript-3ZFPL, 7 μg pBluescript-1ZFPL or pBluescript-2ZFPL are cut with Agel, dephosphorylated, and cut with Spel. Inserts are obtained by applying Spel, SAP, and subsequently Xmal to 10 μg pAN1049 or pAN1073 or pAN1670, respectively. Cloning of pBluescript-6ZFPL was done by treating 14 μg of pBluescript-3ZFPL with Agel, SAP, and thereafter Spel to obtain cut vectors. 3ZFPL inserts were released from 20 μg of pBluescript-3ZFPL by incubating with Spel, SAP, and subsequently Xmal.

Ligation reactions for libraries containing one, two, and three ZFPs were set up in a 3:1 molar ratio of insert:vector using 200 ng cut vector, 400 U T4 DNA ligase in 20 μl total volume at RT (room temperature) overnight. Ligation reactions of hexameric zinc finger protein libraries included 2000 ng pBluescript-3ZFPL, 500 ng 3ZFPL insert, 4000 U T4 DNA ligase in 200 μl total volume, which were divided into ten times 20 μl and incubated separately at RT overnight. Portions of ligation reactions were transformed into Escherichia coli by several methods depending on the number of clones required for each library. For generation of pBluescript-1ZFPL and pBluescript-2ZFPL, 3 μl of ligation reaction were directly used for heat shock transformation of E. coli NEB 5-alpha. Plasmid DNA of ligation reactions of pBluescript-3ZFPL was purified using NucleoSpin Gel and PCR Clean-up kit and transformed into electrocompetent E. coli NEB 5-alpha (EasyjecT Plus electroporator from EquiBio or Multiporator from Eppendorf, 2.5 kV and 25 μF, 2 mm electroporation cuvettes from Bio-Rad). Ligation reactions of pBluescript-6ZFP libraries were applied to NucleoSpin Gel and PCR Clean-up kit and DNA was eluted in 15 μl of deionized water. About 60 ng of desalted DNA were mixed with 50 μl NEB 10-beta electrocompetent E. coli (New England Biolabs) and electroporation was performed as recommended by the manufacturer using EasyjecT Plus or Multiporator, 2.5 kV, 25 μF and 2 mm electroporation cuvettes. Multiple electroporations were performed for each library and cells were directly pooled afterwards to increase library size. After heat shock transformation or electroporation, SOC medium was applied to the bacteria and after 1 h of incubation at 37° C. and 250 rpm, 30 μl of SOC culture were used for serial dilutions and plating on LB plates containing ampicillin. The next day, total number of obtained library clones was determined. In addition, ten clones of each library were chosen to isolate plasmid DNA and to check incorporation of inserts by restriction enzyme digestion. At least three of these plasmids were sequenced to verify diversity of the library. The remaining SOC culture was transferred to 100 ml LB medium containing ampicillin and cultured overnight at 37° C. and 250 rpm. Those cells were used to prepare plasmid Midi DNA for each library.

For yeast one hybrid screens, hexameric zinc finger protein libraries are transferred to a compatible prey vector. For that purpose, the multiple cloning site of pGAD10 (Clontech) was modified by cutting the vector with Xhol/EcoRI and inserting annealed oligonucleotides OAN971 (TCGACAGGCCCAGGCGGCCCTCGAGGATATCATGATG ACTAGTGGCCAGGCCGGCCC, SEQ ID NO: 80) and OAN972 (AATTGGGCCGGC CTGGCCACTAGTCATCATGATATCCTCGAGGGCCGCCTGGGCCTG, SEQ ID NO: 81). The resulting vector pAN1025 (SEQ ID NO: 82) was cut and dephosphorylated, 6ZFP library inserts were released from pBluescript-6ZFPL by Xhol/Spel. Ligation reactions and electroporations into NEB 10-beta electrocompetent E. coli were done as described above for pBluescript-6ZFP libraries.

For improved yeast one hybrid screening, hexameric zinc finger libraries are also transferred into an improved prey vector pAN1375 (SEQ ID NO: 83). This prey vector was constructed as follows: pRS315 (SEQ ID NO: 84) was cut ApallNarl and annealed OAN1143 (CGCCGCATGCATTCATGCAGGCC, SEQ ID NO: 85) and OAN1144 (TGCATGAATGCATGCGG, SEQ ID NO: 86) were inserted yielding pAN1373 (SEQ ID NO: 87). A Sphl insert from pAN1025 was ligated into pAN1373 cut with Sphl to obtain pAN1375.

For further improved yeast one hybrid screening, hexameric zinc finger libraries are also transferred into an improved prey vector pAN1920 (SEQ ID NO: 88).

For even further improved yeast one hybrid screening, hexameric zinc finger libraries are inserted into prey vector pAN1992 (SEQ ID NO: 89).

Cloning of Bait Plasmids for Yeast One Hybrid Screening

For each bait plasmid, a 60 bp sequence containing a potential artificial transcription factor target site of 18 bp in the center is selected and a Ncol site is included for restriction analysis. Oligonucleotides are designed and annealed in such a way to produce 5′ HindIII and 3′ Xhol sites which allowed direct ligation into pAbAi (Clontech) cut with HindIII/Xhol. Digestion of the product with Ncol and sequencing are used to confirm assembly of the bait plasmid.

Yeast Strain and Media

Saccharomyces cerevisiae Y1H Gold was purchased from Clontech, YPD medium and YPD agar from Carl Roth. Synthetic drop-out (SD) medium contained 20 g/l glucose, 6.8 g/l Na₂HPO₄.2H₂O, 9.7 g/l NaH₂PO₄.2H₂O (all from Carl Roth), 1.4 g/l yeast synthetic drop-out medium supplements, 6.7 g/l yeast nitrogen base, 0.1 g/l L-tryptophan, 0.1 g/l L-leucine, 0.05 g/l L-adenine, 0.05 g/l L-histidine, 0.05 g/l uracil (all from Sigma-Aldrich). SD-U medium contained all components except uracil, SD-L was prepared without L-leucine. SD agar plates did not contain sodium phosphate, but 16 g/l Bacto Agar (BD). Aureobasidin A (AbA) was purchased from Clontech.

Preparation of Bait Yeast Strains

About 5 μg of each bait plasmid are linearized with BstBl in a total volume of 20 μl and half of the reaction mix is directly used for heat shock transformation of S. cerevisiae Y1H Gold. Yeast cells are used to inoculate 5 ml YPD medium the day before transformation and grown overnight on a roller at RT. One milliliter of this pre-culture is diluted 1:20 with fresh YPD medium and incubated at 30° C., 225 rpm for 2-3 h. For each transformation reaction 1 OD₆₀₀ cells are harvested by centrifugation, yeast cells are washed once with 1 ml sterile water and once with 1 ml TE/LiAc (10 mM Tris/HCl, pH 7.5, 1 mM EDTA, 100 mM lithium acetate). Finally, yeast cells are resuspended in 50 μl TE/LiAc and mixed with 50 μg single stranded DNA from salmon testes (Sigma-Aldrich), 10 μl of BstBl-linearized bait plasmid (see above), and 300 μl PEG/TE/LiAc (10 mM Tris/HCl, pH 7.5, 1 mM EDTA, 100 mM lithium acetate, 50% (w/v) PEG 3350). Cells and DNA are incubated on a roller for 20 min at RT, afterwards placed into a 42° C. water bath for 15 min. Finally, yeast cells are collected by centrifugation, resuspended in 100 μl sterile water and spread onto SD-U agar plates. After 3 days of incubation at 30° C. eight clones growing on SD-U from each transformation reaction are chosen to analyze their sensitivity towards aureobasidin A (AbA). Pre-cultures were grown overnight on a roller at RT. For each culture, OD₆₀₀ was measured and OD₆₀₀=0.3 was adjusted with sterile water. From this first dilution five additional 1/10 dilution steps were prepared with sterile water. For each clone 5 μl from each dilution step were spotted onto agar plates containing SD-U, SD-U 100 ng/ml AbA, SD-U 150 ng/ml AbA, and SD-U 200 ng/ml AbA. After incubation for 3 days at 30° C., three clones growing well on SD-U and being most sensitive to AbA are chosen for further analysis. Stable integration of bait plasmid into yeast genome is verified by Matchmaker Insert Check PCR Mix 1 (Clontech) according to the manufacturer's instructions. One of three clones is used for subsequent Y1H screen.

Transformation of Bait Yeast Strain with Hexameric Zinc Finger Protein Library

About 500 μl of yeast bait strain pre-culture are diluted into 1 I YPD medium and incubated at 30° C. and 225 rpm until OD₆₀₀=1.6-2.0 (circa 20 h). Cells are collected by centrifugation in a swing-out rotor (5 min, 1500×g, 4° C.). Preparation of electrocompetent cells is done according to Benatuil L. et al., 2010, Protein Eng Des Sel 23, 155-159. For each transformation reaction, 400 μl electrocompetent bait yeast cells are mixed with 1 μg prey plasmids encoding 6ZFP libraries and incubated on ice for 3 min. The cell-DNA suspension is transferred to a pre-chilled 2 mm electroporation cuvette. Multiple electroporation reactions (EasyjecT Plus electroporator or Multiporator, 2.5 kV and 25 μF) are performed until all yeast cell suspension has been transformed. After electroporation yeast cells are transferred to 100 ml of 1:1 mix of YPD:1 M sorbitol and incubated at 30° C. and 225 rpm for 60 min. Cells are collected by centrifugation and resuspended in 1-2 ml of SD-L medium. Aliquots of 200 μl are spread on 15 cm SD-L agar plates containing 1000-4000 ng/ml AbA. In addition, 50 μl of cell suspension are used to make 1/100 and 1/1000 dilutions and 50 μl of undiluted and diluted cells are plated on SD-L. All plates are incubated at 30° C. for 3 days. The total number of obtained clones is calculated from plates with diluted transformants. While SD-L plates with undiluted cells indicate growth of all transformants, AbA-containing SD-L plates only resulted in colony formation if the prey 6ZFP bound to its bait target site successfully.

Verification of Positive Interactions and Recovery of 6ZFP-Encoding Prey Plasmids

For initial analysis, forty good-sized colonies are picked from SD-L plates containing the highest AbA concentration and yeast cells were restreaked twice on SD-L with 1000-4000 ng/ml AbA to obtain single colonies. For each clone, one colony is used to inoculate 5 ml SD-L medium and cells are grown at RT overnight. The next day, OD₆₀₀=0.3 is adjusted with sterile water, five additional 1/10 dilutions are prepared and 5 μl of each dilution step are spotted onto SD-L, SD-L 500 ng/ml AbA, 1000 ng/ml AbA, SD-L 1500 ng/ml AbA, SD-L 2000 ng/ml AbA, SD-L 2500 ng/ml AbA, SD-L 3000 ng/ml AbA, and SD-L 4000 ng/ml AbA plates. Clones are ranked according to their ability to grow on high AbA concentration. From best growing clones 5 ml of initial SD-L pre-culture are used to spin down cells and to resuspend them in 100 μl water or residual medium. After addition of 50 U lyticase (Sigma-Aldrich, L2524) cells are incubated for several hours at 37° C. and 300 rpm on a horizontal shaker. Generated spheroblasts are lysed by adding 10 μl 20% (w/v) SDS solution, mixed vigorously by vortexing for 1 min and frozen at −20° C. for at least 1 h. Afterwards, 250 μl A1 buffer from NucleoSpin Plasmid kit and one spatula tip of glass beads (Sigma-Aldrich, G8772) are added and tubes are mixed vigorously by vortexing for 1 min. Plasmid isolation is further improved by adding 250 μl A2 buffer from NucleoSpin Plasmid kit and incubating for at least 15 min at RT before continuing with the standard NucleoSpin Plasmid kit protocol. After elution with 30 μl of elution buffer 5 μl of plasmid DNA are transformed into E. coli DH5 alpha by heat shock transformation. Two individual colonies are picked from ampicillin-containing LB plates, plasmids are isolated and library inserts are sequenced. Obtained results are analyzed for consensus sequences among the 6ZFPs for each target site.

Cloning of a Reporter Plasmid for the Generation of Stable Luciferase/Secreted Alkaline Phosphatase Reporter Cell Lines for Testing Transducible Artificial Transcription Factor Activity

To generate a reporter construct containing Gaussia luciferase under the control of a hybrid CMV/artificial transcription factor target site promoter together with secreted alkaline phosphatase under control of the constitutive CMV promoter, 42 bp containing the artificial transcription factor binding site were cloned AflIII/Spel into pAN1660 (SEQ ID NO: 90). These reporter constructs contain a Flpin site for stable integration into Flpin site containing cells such as HEK 293 Flpin TRex (Invitrogen) cells. Oligonucleotides OAN1612 (SEQ ID NO: 91) and OAN1613 (SEQ ID NO: 92) were used to generate such a reporter construct for testing artificial transcription factors targeting AR_TS4.

Cloning of Artificial Transcription Factors for Mammalian Transfection

DNA fragments encoding polydactyl zinc finger proteins are cloned using standard procedures with Agel/Xhol into mammalian expression vectors for expression in mammalian cells as fusion proteins between the zinc finger array of interest, a SV40 NLS, a 3×myc epitope tag and a N-terminal KRAB domain (pAN1255-SEQ ID NO: 93), a C-terminal KRAB domain (pAN1258-SEQ ID NO: 94), a SID domain (pAN1257-SEQ ID NO: 95) or a VP64 activating domain (pAN1510-SEQ ID NO: 96).

Plasmids for the generation of stably transfected, tetracycline-inducible cells were generated as follows: DNA fragments encoding artificial transcriptions factors comprising polydactyl zinc finger domain, a regulatory domain (N-terminal KRAB, C-terminal KRAB, SID or VP64), and a SV40 NLS are cloned into pAN2071 (SEQ ID NO: 97) using EcoRV/Agel. These artificial transcription factor expression plasmids can be integrated into the human genome into the AAVS1 locus by co-transfection with AAVS1 Left TALEN and AAVS1 Right TALEN (GeneCopoeia).

Cell Culture and Transfections

HeLa cells are grown in Dulbecco's Modified Eagle's Medium (DMEM) supplemented with 4.5 g/l glucose, 10% heat-inactivated fetal bovine serum, 2 mM L-glutamine, and 1 mM sodium pyruvate (all from Sigma-Aldrich) in 5% CO₂ at 37° C. For luciferase reporter assay, 7000 HeLa cells/well are seeded into 96 well plates. Next day, co-transfections are performed using Effectene Transfection Reagent (Qiagen) according to the manufacturer's instructions. Plasmid midi preparations coding for artificial transcription factor and for luciferase are used in the ratio 3:1. Medium is replaced by 100 μl per well of fresh DMEM 6 h and 24 h after transfection.

Generation and Maintenance of Flp-In™ T-Rex™ 293 Expression Cell Lines

Stable, tetracycline inducible Flp-In™ T-Rex™ 293 expression cell lines are generated by Flp Recombinase-mediated integration. Using Flp-In™ T-Rex™ Core Kit, the Flp-In™ T-Rex™ host cell line is generated by transfecting pFRT/lacZeo target site vector and pcDNA6/TR vector. For generation of inducible 293 expression cell lines, the pcDNA5/FRT/TO expression vector containing the gene of interest is integrated via Flp recombinase-mediated DNA recombination at the FRT site in the Flp-In™ T-Rex™ host cell line. Stable Flp-In™ T-Rex™ expression cell lines are maintained in selection medium containing (DMEM; 10% Tet-FBS; 2 mM glutamine; 15 μg/ml blasticidine and 100 μg/ml hygromycin). For induction of gene expression tetracycline is added to a final concentration of 1 μg/mL.

Generation and Maintenance of Stably Artificial Transcription Factor Expressing Cell Lines Using TALENs

To generate cell lines stably expressing artificial transcription factors under the control of a tetracycline-inducible promoter, cells are co-transfected with a pAN2071-based expression construct containing the artificial transcription factor of interest and AAVS1 Left TALEN and AAVS1 Right TALEN (GeneCopoeia) plasmids using Effectene (Qiagen) transfection reagent) according to the manufacturer's recommendations. 8 hours post-transfection, growth medium was aspirated, cells were washed with PBS and fresh growth medium was added. 24 h post transfection cells were split at a ratio of 1:10 in growth medium containing Tet-approved FBS (tetracycline free FBS, Takara) without antibiotics. 48 h post-transfection, puromycin selection was started at cell-type specific concentration and cells were kept under selection pressure for 7-10 days. Colonies of stable cells were pooled and maintained in selection medium.

Determination of Gene Expression Levels by Quantitative RT-PCR

Total RNA is isolated from cells using the RNeasy Plus Mini Kit (Qiagen, Hilden, Germany) according to the manufacturer's instructions. Frozen cell pellets are resuspended in RLT Plus Lysis buffer containing 10 μl/ml β-mercaptoethanol. After homogenization using QIAshredder spin columns, total lysate is transferred to gDNA Eliminator spin columns to eliminate genomic DNA. One volume of 70% ethanol is added and total lysate is transferred to RNeasy spin columns. After several washing steps, RNA is eluted in a final volume of 30 μl RNase free water. RNA is stored at −80° C. until further use. Synthesis of cDNA is performed using the High Capacity cDNA Reverse Transcription Kit (Applied Biosystems, Branchburg, N.J., USA) according to the manufacturer's instructions. cDNA synthesis is carried out in 20 μl of total reaction volume containing 2 μl 10× Buffer, 0.8 μl 25×dNTP Mix, 2 μl 10×RT Random Primers, 1 μl Multiscribe Reverse Transcriptase and 4.2 μl H₂O. A final volume of 10 μl RNA is added and the reaction is performed under the following conditions: 10 minutes at 25° C., followed by 2 hours at 37° C. and a final step of 5 minutes at 85° C. Quantitative PCR is carried out in 20 μl of total reaction volume containing 1 μL 20×TaqMan Gene Expression Master Mix, 10.0 μl TaqMan® Universal PCR Master Mix (both Applied Biosystems, Branchburg, N.J., USA) and 8 μl H₂O. For each reaction 1 μl of cDNA is added. qPCR is performed using the ABI PRISM 7000 Sequence Detection System (Applied Biosystems, Branchburg, N.J., USA) under the following conditions: an initiation step for 2 minutes at 50° C. is followed by a first denaturation for 10 minutes at 95° C. and a further step consisting of 40 cycles of 15 seconds at 95° C. and 1 minute at 60° C.

Cloning of Artificial Transcription Factors for Bacterial Expression

DNA fragments encoding artificial transcription factors are cloned using standard procedures with EcoRV/Notl into bacterial expression vector pAN983 (SEQ ID NO: 98) based on pET41a+ (Novagen) for expression in E. coli as His₆-tagged fusion proteins between the artificial transcription factor and the TAT protein transduction domain.

Expression constructs for the bacterial production of transducible artificial transcription factors in suitable E. coli host cells such as BL21(DE3) targeting GR, AR, or ER are pAN2343 (SEQ ID NO: 99), pAN2344 (SEQ ID NO: 100), pAN2345 (SEQ ID NO: 101), pAN2346 (SEQ ID NO: 102), and pAN2347 (SEQ ID NO: 103).

Production of Artificial Transcription Factor Protein

E. coli BL21(DE3) transformed with expression plasmid for a given artificial transcription factor were grown in 1 I LB media supplemented with 100 μM ZnCl₂ until OD₆₀₀ between 0.8 and 1 was reached, and induced with 1 mM IPTG for two hours. Bacteria were harvested by centrifugation, bacterial lysate was prepared by sonication, and inclusion bodies were purified. To this end, inclusion bodies were collected by centrifugation (5000 g, 4° C., 15 minutes) and washed three times in 20 ml of binding buffer (50 mM HEPES, 500 mM NaCl, 10 mM imidazole; pH 7.5). Purified inclusion bodies were solubilized on ice for one hour in 30 ml of binding buffer A (50 mM HEPES, 500 mM NaCl, 10 mM imidazole, 6 M GuHCI; pH 7.5). Solubilized inclusion bodies were centrifuged for 40 minutes at 4° C. and 13′000 g and filtered through 0.45 μm PVDF filter. His-tagged artificial transcription factors were purified using His-Trap columns on an Äktaprime FPLC (GEHealthcare) using binding buffer A and elution buffer B (50 mM HEPES, 500 mM NaCl, 500 mM imidazole, 6 M GuHCI; pH 7.5). Fractions containing purified artificial transcription factor were pooled and dialyzed at 4° C. overnight against buffer S (50 mM Tris-HCl, 500 mM NaCl, 200 mM arginine, 100 μM ZnCl₂, 5 mM GSH, 0.5 mM GSSG, 50% glycerol; pH 7.5) in case the artificial transcription factor contained a SID domain, or against buffer K (50 mM Tris-HCl, 300 mM NaCl, 500 mM arginine, 100 μM ZnCl₂, 5 mM GSH, 0.5 mM GSSG, 50% glycerol; pH 8.5) for KRAB domain containing artificial transcription factors. Following dialysis, protein samples were centrifuged at 14′000 rpm for 30 minutes at 4° C. and sterile filtered using 0.22 μm Millex-GV filter tips (Millipore). For artificial transcription factors containing VP64 activation domain, the protein was produced from the soluble fraction (binding buffer: 50 mM NaPO₄ pH 7.5, 500 mM NaCl, 10 mM imidazole; elution buffer 50 mM HEPES pH 7.5, 500 mM NaCl, 500 mM imidazole) using His-Bond Ni-NTA resin (Novagen) according to manufactures recommendation. Protein was dialyzed against VP64-buffer (550 mM NaCl pH 7.4, 400 mM arginine, 100 μM ZnCl₂).

Protein Transduction

Cells grown to about 80% confluency are treated with 0.01 to 1 μM artificial transcription factor or mock treated for 2 h to 120 h with optional addition of artificial transcription factor every 24 h in OptiMEM or growth media at 37° C. Optionally, 10-500 μM ZnCl₂ are added to the growth media. For immunofluorescence, cells are washed once in PBS, trypsinized and seeded onto glass cover slips for further examination.

Immunofluorescence

Cells are fixed with 4% paraformaldehyde, treated with 0.15% Triton X-100, blocked with 10% BSA and incubated overnight with mouse anti-HA antibody (1:500, H9658, Sigma) or mouse anti-myc (1:500, M5546, Sigma). Samples are washed three times with PBS/1% BSA, and incubated with goat anti-mouse antibodies coupled to Alexa Fluor 546 (1:1000, Invitrogen) and counterstained using DAPI (1:1000 of 1 mg/ml for 3 minutes, Sigma). Samples are analyzed using fluorescence microscopy.

Combined Luciferase/SEAP Promoter Activity Assay

To test activity of artificial transcription factors, a reporter cell line was employed. This reporter cell line is based on HEK 293 Flpin TRex cells containing Gaussia luciferase under control of a hybrid CMV/artificial transcription factor target site promoter and secreted alkaline phosphatase under control of a constitutive CMV promoter.

1×10⁵ reportercells/well are seeded in 6-well plates 24 h before protein transduction. 24 h after seeding, medium is aspirated from the plate and cells are washed 1× with PBS. For protein treatment, AR4rep was diluted to a final concentration of 1 μM in OptiMEM, added to the cells and incubated for 2 h in an incubator (37° C.; 5% CO₂). Following protein transduction, cells were grown for 24 h in normal growth medium. Supernatant was transferred to 96 well plates, and centrifuged at 2000 rpm for 5 min. For measurement of Gaussia Luciferase the Pierce™ Gaussia Luciferase Glow Assay Kit (Thermo Scientific) was used according to manufacturer's instructions. The working solution was equilibrated to room temperature and coelenterazine was added at a dilution of 1:100. 20 μl of cell supernatant was transferred into an opaque 96-well plate and 50 μl of working solution was added. After 10 min of incubation luminescence was measured using MicroLumatPlus (Berthold Technologies) at an integration time of 1.0 s. For measurement of secreted alkaline phosphatase activity the chemiluminescent SEAP Reporter Gene Assay (Roche) was used according to manufacturer's instructions. Cell supernatant was diluted 1:4 with dilution buffer and heat inactivated at 65° C. for 5 min. 50 μL of heat inactivated sample was transferred to a an opaque 96-well plate and 50 μL of inactivation buffer was added. After incubation for 5 min at room temperature, 50 μL of substrate reagent, consisting of AP Substrate 1:20 in substrate buffer, was added and incubated for 10 min at room temperature under gentle agitation. Luminescence was measured using MicroLumatPlus (Berthold Technologies) at an integration time of 1.0 s.

1-21. (canceled)

22. An artificial transcription factor comprising a polydactyl zinc finger protein targeting specifically a promoter region of a nuclear receptor gene fused to an inhibitory or activatory protein domain, a nuclear localization sequence, and a protein transduction domain.

23. An artificial transcription factor according to claim 22, wherein the promoter region of the nuclear receptor gene is the androgen receptor promoter.

24. An artificial transcription factor according to claim 22, wherein the promoter region of the nuclear receptor gene is the estrogen receptor promoter.

25. The artificial transcription factor according to claim 22 comprising a hexameric zinc finger protein.

26. The artificial transcription factor according to claim 22 wherein the zinc finger protein is fused to an inhibitory protein domain.

27. The artificial transcription factor according to claim 26 wherein the inhibitory protein domain is N-terminal KRAB of SEQ ID NO: 1, C-terminal KRAB of SEQ ID NO: 2, SID of SEQ ID NO: 3, or ERD of SEQ ID NO: 4.

28. The artificial transcription factor according to claim 22 wherein the zinc finger protein is fused to an activatory protein domain.

29. The artificial transcription factor according to claim 28 wherein the activatory protein domain is VP16 of SEQ ID NO: 5, VP64 of SEQ ID NO: 6, CJ7 of SEQ ID NO: 7, p65TA1 of SEQ ID NO: 8, SAD of SEQ ID NO: 9, NF-1 of SEQ ID NO: 10, AP-2 of SEQ ID NO: 11, SP1-A of SEQ ID NO: 12, SP1-B of SEQ ID NO: 13, Oct-1 of SEQ ID NO: 14, Oct-2 of SEQ ID NO: 15, Oct2-5x of SEQ ID NO: 16, MTF-1 of SEQ ID NO: 17, BTEB-2 of SEQ ID NO: 18 or LKLF of SEQ ID NO: 19.

30. The artificial transcription factor according to claim 22, wherein the nuclear localization sequences is a cluster of basic amino acids containing the K-K/R-X-K/R consensus sequence or the SV40 NLS of SEQ ID NO: 75.

31. The artificial transcription factor according to claim 22, wherein the protein transduction domain is the HIV derived TAT peptide of SEQ ID NO: 20, the synthetic peptide mT02 of SEQ ID NO: 25, the synthetic peptide mT03 of SEQ ID NO: 26, the R9 peptide of SEQ ID NO: 27, or the ANTP domain of SEQ ID NO: 28.

32. The artificial transcription factor according to claim 22 comprising a zinc finger protein of a protein sequence selected from the group consisting of SEQ ID NO: 39 to 41, 48 to 53, and 66 to 68.

33. An artificial transcription factor comprising a polydactyl zinc finger protein targeting specifically a promoter region of a nuclear receptor gene fused to an inhibitory or activatory protein domain, and a nuclear localization sequence.

34. The artificial transcription factor according to claim 22 further comprising a polyethylene glycol residue.

35. A pharmaceutical composition comprising an artificial transcription factor according to claim 22.

36. An E. coli host cell containing an expression construct of SEQ ID NO: 99 to 103 for the production of the artificial transcription factor of claim 22.

37. The artificial transcription factor according to claim 22 for use in modulating the cellular response to ligands of nuclear receptors.

38. A method of treatment of a disease, wherein modulation of expression of a nuclear receptor gene is therapeutically beneficial, comprising administering a therapeutically effective amount of an artificial transcription factor according to claim 22 to a patient in need thereof.

39. A method of treatment according to claim 38, wherein the nuclear receptor is the androgen receptor, and the disease is modulated by testosterone and selected from the group consisting of cancer, coronary artery disease, obesity, diabetes, schizophrenia, depression and attention deficit hyperactivity disorder.

40. A method of treatment according to claim 38, wherein the nuclear receptor is the estrogen receptor, and the disease is modulated by estrogens and selected from the group consisting of cancer, cardiovascular disease, osteoporosis and mood disorders.

41. A method of treatment according to claim 38, wherein the nuclear receptor is the glucocorticoid receptor, and the disease is modulated by glucocorticoids and selected from the group consisting of inflammatory processes, diabetes, obesity, coronary artery disease, asthma, celiac disease and lupus erythematosus.
